(19) United States
(12) Patent Application Publication
(10) Pub. No.: US 2006/0159737 A1
(43) Pub. Date: Jul. 20, 2006

(54) Pharmaceutical compositions for local administration

(76) Inventors: Steffen Panzner, Halle (DE); Cornelia Panzner, Halle (DE); Silke Lutz, Halle (DE); Gerold Endert, Halle (DE)

Correspondence Address: MINTZ LEVIN COHN FERRIS GLOVSKY & POPEO, 666 THIRD AVENUE, NEW YORK, NY, 10017, US

(21) Appl. No.: 11/267,423

(22) Filed: Nov. 4, 2005

Related U.S. Application Data
(60) Provisional application No. 60/629,600, filed on Nov. 19, 2004.
(60) Provisional application No. 60/717,199, filed on Sep. 15, 2005.

(30) Foreign Application Priority Data
Nov. 19, 2004 (DE) 10 2004 056 659.3
Sep. 15, 2005 (EP) EP 05 020 217.5

(51) Int. Cl. A61K 48/00; A61K 9/127; C12N 15/88

(52) U.S. Cl. 424/450; 514/44; 435/458

(57) ABSTRACT

A pharmaceutical composition for local application is disclosed, said composition comprising a nucleic acid as a therapeutic agent, an excipient and a pharmaceutically acceptable vehicle therefor, said excipient comprising a liposome. The excipient comprises an amphoteric liposome having an isoelectric point between 4 and 7.4 and said composition is formulated to have a pH in the range 3 to 5. The composition may administered in the form of a colloidal suspension and may be buffered to the lower pH at the time of use by the addition of a suitable acidifying means to a substantially neutral suspension of the nucleic acid and excipient that may be more suitable for long-term storage of the composition. Alternatively, the composition may be lyophilised at the lower pH for subsequent reconstitution just prior to use with a suitable aqueous medium, such for example as substantially unbuffered water or saline.

This application claims priority to German Patent Application No. DE 10 2004 056 659.3, filed Nov. 19, 2004; European Patent Application No. EP 05 020 217.5, filed Sep. 15, 2005. The application also claims the benefit of U.S. Patent Application Ser. No. No. 60/629,600, filed Nov. 19, 2004 and U.S. Patent Application Ser. No. 60/717,199, filed Sep. 15, 2005, the entire contents of which are hereby incorporated by reference.

### FIELD OF THE INVENTION

The present invention relates to pharmaceutical compositions for local administration to a human or non-human animal or to grafts for transplant, and has particular reference to such compositions which comprise a nucleic acid as a therapeutic agent. The present invention also comprehends the use of such a composition in the manufacture of a medicament for local administration. The present invention embraces methods of treatment or prophylaxis of inflammatory, immune or autoimmune disorders using nucleic acid therapeutics and kits for formulating a composition in accordance with the invention at the time of use.

### BACKGROUND OF THE INVENTION

Nucleic acid therapeutics represent a new class of drugs for systemic or local administration. Excluding CpG-oligos or aptamers, the majority of such therapeutics have an intracellular site of action and can be classified into nucleic acids encoding one or more specific protein, polypeptides or RNA sequences and oligonucleotides that can specifically down-regulate protein expression.

Oligonucleotides include antisense, locked nucleic acids (LNA), peptide nucleic acids (PNA), morpholino nucleic acids (Morpholinos), small interfering RNAs (siRNA) and decoys of various chemistries. A detailed description of the different mechanisms can be found in the literature (e.g. Crooke in BBA (1999), 1489(1), 31-44; Tijsterman, et al. in Cell (2004), 117(1), 1-3; or Mann, et al. in J Clin Invest, (2000), 106(9), 1071-5). Nucleic acid therapeutics have been proposed for the treatment of a variety of diseases. In addition to systemic application, there are many preclinical and clinical studies, especially in the area of inflammatory or immune-mediated diseases and disorders and in the field of genetic vaccination, that deal with the local application of such drugs to mucous membranes, ex vivo to grafts and to the eyes (e.g. Shanahan in Expert Opin Investig Drugs, (1999), 8(9), 1417-1429; Ball, et al. in Am J Pharmacogenomics, (2003), 3(2), 97-106; Finotto, et al. in J Allergy Clin Immunol., (2002), 107(2), 279-286; Nedbal, et al. in Antisense Nucleic Acid Drug Dev., (2002), 12(2), 71-78; Bochot, et al. in Prog Retin Eye Res., (2000), 19(2), 131-147; Rogy, et al. in Human Gene Therapy, (2000), 11(12), 1731-1741; Klavinskis in J. Immunol. (1999), 162, 254-262; Hopson, et al. in Methods (2003), 31(3), 217-224; and Barnes, et al. in Curr Opin Mol Ther. (2000), 2(1), 87-93.

It is known in the art that nucleic acid therapeutics, irrespective of their actual chemical origin, may lack therapeutic efficacy owing to their instability in body fluids or inefficient uptake into cells, or both. The chemical modification of such oligonucleotides, including those referred to above as wells as conjugation with ligands or polymers, represents one strategy for overcoming such practical limitations.

A second approach comprehends the use of a carrier system such, for example, as a liposome for the protection, targeting or enhanced uptake of the nucleic acid into cells. For use as such a carrier system, a liposome should desirably show a high encapsulation efficiency and be economical to produce; it should have a good colloidal stability and provide an enhanced uptake of the drug into cells; it should also have a low toxicity and immunogenicity.

Anionic or neutral liposomes often possess excellent colloidal stability, since substantially no aggregation occurs between the carrier and the environment. Consequently their biodistribution may be excellent, and their potential for irritation and cytotoxicity is low. However, such carriers often lack encapsulation efficiency and do not provide an endosomolytic signal that may facilitate the further uptake into cells (Journal of Pharmacology and experimental Therapeutics (2000), 292, 480488 by Klimuk, et al.).

A great many publications deal with cationic liposomal systems, e.g. Molecular Membrane Biology (1999), 16, 129-140 by Maurer, et al.; BBA (2000) 1464, 251-261 by Meidan, et al.; Reviews in Biology and Biotechnology (2001), 1(2), 27-33 by Fiset & Gounni. Although cationic systems may provide high loading efficiencies, they often lack colloidal stability, especially after contact with body fluids. Ionic interactions with proteins or other biopolymers may lead to the formation of aggregates with the extracellular matrix or with cell surfaces in situ. Cationic lipids have also often been found to be toxic, as shown for nstance by Filion, et al. in BBA (1997), 1329(2), 345-356; Dass in J. Pharm. Pharmacol. (2002), 54(5), 593-601; and Hirko, et al. in Curr. Med. Chem., 10(14), 1185-1193. Such limitations may be overcome by the addition of components that provide steric stabilisation of the carrier. Polyethylenglycols of various chain length, for example, are known to reduce the aggregation problems associated with the use of cationic components in body fluids, and PEGylated cationic liposomes may show enhanced circulation times in vivo (BBA (2001) 1510, 152-166 by Semple, et al.). However, the use of PEG does not solve the intrinsic toxicity problem associated with cationic lipids. It is also known that PEG may substantially inhibit the productive entry of such liposomes into cells or their intracellular delivery (Song, et al. in BBA (2002), 1558(1), 1-13).

Amphoteric liposomes represent a recently described class of liposomes having an anionic or neutral charge at pH 7.4 and a cationic charge at pH 4. Reference is made here to WO 02/066490, WO 02/066012 and WO 03/070735, all to Panzner, et al. which give a detailed description of certain amphoteric liposomes and which are incorporated herein by reference. Further disclosures are made in WO 03/070220 and WO 03 070735, also to Panzner, et al. and incorporated herein by reference, describing more pH sensitive lipids for the manufacture of amphoteric liposomes. Amphoteric liposomes have been found to have a good biodistribution and to be well tolerated in animals; they can encapsulate nucleic acid molecules with high efficiency.

### OBJECT OF THE INVENTION

An object of the present invention is to provide a pharmaceutical composition comprising a nucleic acid therapeutic for local application to a mucous membrane, ex vivo to a graft before transplantation or to the eye.

Another object of the present invention is to provide a method for the treatment or prophylaxis of an inflammatory or immune-mediated disease or disorder by local administration of a pharmaceutical composition in accordance with the invention.

### SUMMARY OF THE INVENTION

According to one aspect of the present invention therefore there is provided a pharmaceutical composition for local administration, said composition comprising a nucleic acid as a therapeutic agent, an excipient and a pharmaceutically acceptable vehicle therefor, said excipient comprising a liposome; characterised in that said excipient comprises an amphoteric liposome having an isoelectric point between about 4 and about 7.4 and said composition is formulated to have a pH in the range of about 3 to about 5.

In some embodiments, the excipient may have an isoelectric point of less than 7. The composition may be formulated to have a pH in the range 4 to 6, preferably pH 4 to 5.

Said composition may be administered in the form of a suspension, particularly a colloidal suspension and may therefore be buffered to the lower pH at the time of use by the addition of a suitable acidifying means to a substantially neutral suspension of the nucleic acid and excipient that may be more suitable for long-term storage of the composition. Alternatively, the composition according to the invention may be lyophilised at the lower pH for subsequent reconstitution just prior to use with a suitable aqueous medium, such for example as substantially unbuffered water or saline.

Thus, in another aspect of the present invention there is provided a kit comprising a pharmaceutical composition and instructions for the use thereof, said composition comprising a nucleic acid as a therapeutic agent, an excipient and a pharmaceutically acceptable vehicle therefor, which excipient comprises a liposome, characterised in that said excipient comprises an amphoteric liposome having an isoelectric point between 4 and 7.4 and in that said composition is provided in the form of a suspension at substantially neutral pH, said instructions directing acidification of said suspension prior to use to a pH in the range of about 3 to about 5, and in an alternative aspect of the present invention there is provided a kit comprising a pharmaceutical composition and instructions for the use thereof, said composition comprising a nucleic acid as a therapeutic agent, an excipient and a pharmaceutically acceptable vehicle therefor, which excipient comprises a liposome, characterised in that said excipient comprises an amphoteric liposome having an isoelectric point of between 4 and 7.4 and in that said composition is provided in lyophilised form such that upon reconstitution with an aqueous medium the pH of the reconstituted composition is in the range of about 3 to about 5, said instructions directing the reconstitution of the lyophilised composition at the time of use.

In a different aspect of the present invention, there is provided a method of treatment or prophylaxis of an inflammatory, immune or autoimmune disorder comprising administering a pharmaceutically or prophylactically amount of a pharmaceutical composition in accordance with the present invention to a human or non-human animal patient in need thereof, wherein said therapeutic agent is adapted to alleviate, prevent or reduce the severity of said inflammatory, immune or autoimmune disorder. In some embodiments, the composition may be administered locally to a mucous membrane, for example such a membrane in the nose, airway, mouth, intestine or vagina, or to the eye. The composition may be applied topically.

Suitably, said nucleic acid may comprise an oligonucleotide that is adapted to target nucleic acids encoding CD40, thereby to modulate the expression of CD40 in mammalian cells. Preferably, said oligonucleotide is directed against human CD40. As described in co-pending application number PCT/EP05/nnnnn, filed on 4 Nov. 2005 (attorney docket no. 33841-501-WO1), the contents of which are incorporated herein by reference, CD40 represents an attractive target for the treatment of inflammatory or immune disorders which potentially can be alleviated using oligonucleotide inhibitors such, for example, as antisense or siRNA molecules.

In yet another aspect of the present invention, there is provided a method for treating a graft prior to transplantation, which method comprises administering to said graft ex vivo a pharmaceutical composition in accordance with the present invention. In some embodiments, said composition may comprise a nucleic acid therapeutic that is adapted to prevent or reduce the severity of the symptoms of graft rejection or graft-v-host disease.

In yet another aspect of the present invention there is provided method of vaccinating a human or non-human animal with a genetic vaccine, which method comprising administering an effective amount of a pharmaceutical composition in accordance with the invention.

The present invention is therefore directed to pharmaceutical compositions comprising amphoteric liposomes and nucleic acid therapeutics, which compositions can be locally administered to mucous membranes, to the eyes or ex vivo to grafts. A substantial proportion, or all of the nucleic acid therapeutic, may be physically entrapped within the amphoteric liposomes. Preferably the amphoteric liposome is stable at slightly acidic pHs.

The pharmaceutical composition of the present invention may also be used for other topical treatments of conditions or diseases in mammals or of parts of mammals, especially humans or their organs.

### BRIEF DESCRIPTION OF THE DRAWINGS

FIG. 1: POPC content was increased within the DOTAP/CHEMS mixture. At least 40% of POPC are needed to completely prevent particle growth at low pH.

FIG. 2: Liposomes were produced at pH 7.5 and adjusted to acidic conditions to promote aggregation. Addition of 20mol % POPC greatly reduces the fusion tendency

FIG. 3: Same as in (FIG. 2) but DOPE was tested for stabilization. Particle growth starts at a lower pH when DOTAP/CHEMS 25/75 and DOPE/DOTAP/CHEMS 20/20/60 are compared. Still, all mixtures tested undergo strong aggregation and fusion

FIG. 4: Microscopic scoring of colonic damage.

| | |
|---|---|
| Control | control animals, PBS treated |
| CD40/0 | treated at day0, 4 h prior induction |
| CD40/0_3 | treated at day0, 4k prior induction and day3 |
| SCR/0 | treated with scrambled control, 4 h prior induction |
| CD40/3 | treated at day 3 only |
| SCR/3 | treated with scrambled control at day 3 |

FIGS. 5A-D: Colon sections after various treatments.

| | |
|---|---|
| A | normal, unaffected bowel wall |
| B | inflamed, but untreated bowel wall |
| C | treatment prior colitis induction using the scrambled control |
| D | treatment prior colitis induction using the specific CD40 antisense |

FIG. 6: Porcine CD40 cDNA sequence (SEQ ID NO:4) for targeting in accordance with the present invention.

### DETAILED DESCRIPTION OF THE INVENTION

The amphoteric liposomes included as the excipient in the pharmaceutical composition of the present invention may formed from a lipid phase comprising an amphoteric lipid, or a mixture of lipid components with amphoteric properties, and a neutral phospholipid.

By “amphoteric” herein is meant that the liposomes comprise charged groups of both anionic and cationic character wherein:

(i) at least one of the charged groups has a pK between 4 and 7.4,

(ii) the cationic charge prevails at pH 4, and

(iii) the anionic charge prevails at pH 7.4,

whereby the liposomes have an isoelectric point of zero net charge between pH 4 and pH 7.4. Amphoteric character is by this definition different from zwitterionic character, because zwitterions do not have a pK in the range mentioned above. In consequence, zwitterions are essentially neutral over a range of pH values.

Said neutral phospholipid may comprise a phosphatidylcholine or a mixture of phosphatidylcholine and phosphatidylethanolamine. Phosphatidylcholines and phosphatidylethanolamines are neutral lipids with zwitterionic character.

Said neutral phosphatidylcholines or mixture of phosphatidylcholines and phosphatidylethanolamines may be present in the lipid phase to at least 20 mol. %, preferably to at least 25 mol. % or 30 mol. %, and more preferably to more than 40 mol. %.

In some embodiments, said phosphatidylcholine may selected from the group consisting of POPC, natural or hydrogenated soy bean PC, natural or hydrogenated egg PC, DMPC, DPPC or DOPC. (A glossary of the abbreviated forms of the names of lipids used herein is included below for ease of reference. In some cases such abbreviations are those that are commonly used by those skilled in the art.)

Presently preferred phosphatidylcholines are POPC, non-hydrogenated soy bean PC and non-hydrogenated egg PC.

The phosphatidylethanolamine may be selected from the group consisting of DOPE, DMPE and DPPE.

Most preferably said neutral lipid comprises DOPE and POPC, soy bean PC or egg PC.

The lipid phase may comprise an amphoteric lipid. Suitable amphoteric lipids are disclosed in WO 02/066489 as well as in WO 03/070735, the contents of both of which are incorporated herein by reference. Preferably, said amphoteric lipid is selected from the group consisting of HistChol, HistDG, isoHistSuccDG, Acylcarnosin and HCCHol.

Most preferably the amphoteric lipid is HistChol.

The content of amphoteric lipids may be between 5 mol. % and 30 mol. %, preferably from 10-25 mol. %.

Alternatively, the lipid phase may be formulated using pH-responsive anionic and/or cationic components, as disclosed in WO 02/066012, the contents of which are incorporated by reference herein. Cationic lipids sensitive to pH are disclosed in WO 02/066489 and WO 03/070220, the contents of both of which are incorporated by reference herein, and in the references made therein, especially Budker, et al. 1996, Nat Biotechnol. 14(6):760-4, and can be used in combination with constitutively charged anionic lipids or with anionic lipids that are sensitive to pH. Conversely, the cationic charge may also be introduced from constitutively charged lipids that are known to those skilled in the art in combination with a pH sensitive anionic lipid.

Preferred cationic components are DPIM, CHIM, DORIE, DDAB, DAC-Chol, TC-Chol, DOTMA, DOGS, (C18)₂Gly⁺ N,N-dioctadecylamido-glycin, CTAB, CPyC, DODAP and DOEPC.

Particularly preferred cationic lipids are DMTAP, DPTAP, DOTAP, DC-Chol, MoChol and HisChol.

The amphoteric mixtures further comprise anionic lipids, either constitutively or conditionally charged in response to pH, and such lipids are also known to those skilled in the art. Preferred lipids for use with the invention are DOGSucc, POGSucc, DMGSucc, DPGSucc, DMPS, DPPS, DOPS, POPS, DMPG, DPPG, DOPG, POPG, DMPA, DPPA, DOPA, POPA, CHEMS and CetylP.

Particularly preferred anionic lipids are DOGSucc, DMGSucc, DMPG, DPPG, DOPG, POPG, DMPA, DPPA, DOPA, POPA, CHEMS and CetylP.

In some embodiments, said cationic lipids may comprise one or more of DOTAP, DC-Chol, MoChol and HisChol Said anionic lipids may comprise one or more of DMGSucc, DOGSucc, DOPA, CHEMS and CetylP.

In order improve the bioadhesion of amphoteric liposomes to mucous membranes upon local application, it has been found to be advantageous according to the present invention for the liposomes to have a cationic surface charge. Amphoteric liposomes are cationic at a slightly acidic pH, more precisely at a pH below the isoelectric point of the liposome. When administered at such a pH, the amphoteric liposomes should desirably not aggregate or fuse. Such aggregation or fusion of amphoteric liposomes at an acidic pH may depend upon the lipid composition of the liposome and upon the presence of cargo. It has been found, for example, that specific empty and drug-loaded amphoteric liposomes are stable upon a pH-shift to 4-5.

It has been found that amphoteric liposomes in accordance with the present invention may be stable both at pH 7.5 as well as at pH 4-5, and that the local administration of antisense loaded amphoteric liposomes at pH 4-5 may be particularly effective in the treatment of inflammatory diseases or immune-related disorders.

It has also been found that nucleic acid loaded amphoteric liposomes can be lyophilized at pH 4-5. Thus, amphoteric liposomes may provide means for both providing a stable storage form, as well as facilitating effective drug application.

For example, amphoteric liposomes comprising the charged lipids DOTAP and CHEMS have been found to be stable at an acidic pH when the neutral lipid POPC is also present in the bilayer. By “stable” here is meant that the liposomes do not aggregate upon acidification. In contrast, the replacement of POPC with DOPE may leads to destabilisation of the membrane at low pHs. Such destabilisation has also been found for a range of cation:anion ratios in the mixture.

Advantageously, therefore, said lipid phase may comprise POPC, DOTAP and CHEMS, the lipid phase comprising a greater molar amount of CHEMS than DOTAP. In some embodiments of the invention, the lipid phase may comprise 20-60 mol. % POPC, 10-40 mol. % DOTAP and 20-70 mol. % CHEMS, the total being 100 mol. %.

In one preferred embodiment, the lipid phase may comprise about 60 mol. % POPC, about 10 mol. % DOTAP and about 30 mol. % CHEMS, the total being 100 mol. %.

MoChol and CHEMS may also form stable bilayers with POPC. The amount of MoChol in the lipid phase may be substantially equal to or exceed the molar amount of CHEMS. The total molar amount of CHEMS and MoCHOL may between about 30 and about 80 mol. % of the lipid phase.

In one preferred embodiment, the lipid phase may therefore comprise about 30 mol. % POPC, about 35 mol. % MoChol and about 35 mol. % CHEMS, the total being 100 mol. %.

Advantageously, said lipid phase further comprising DOPE.

Thus in another preferred embodiment, said lipid phase comprises about 15 mol. % POPC, about 45 mol. % DOPE, about 20 mol. % MoChol and about 20 mol. % CHEMS, the total being 100 mol. %.

In yet another presently preferred embodiment, said lipid phase comprises about 6 mol. % POPC, about 24 mol. % DOPE, about 46 mol. % MoChol and about 23 mol. % CHEMS, the total being 100 mol. %.

In some embodiments, said lipid phase may comprise POPC, DOPE, MoChol and DMGSucc. The lipid phase may comprise MoChol in greater or substantially equal molar amounts than DMG-Succ; the total molar amount of DMG-Succ and MoChOL may between 30 and 80 mol. % of the lipid phase.

Thus in yet another preferred embodiment, said lipid phase comprises about 15 mol. % POPC, about 45 mol. % DOPE, about 20 mol. % MoChol and about 20 mol. % DMG-Succ, the total being 100 mol. %.

In yet another preferred embodiment, said lipid phase comprises about 6 mol. % POPC, about 24 mol. % DOPE, about 46 mol. % MoChol and about 23 mol. % DMGSucc, the total being 100 mol. %.

In some embodiments, the lipid phase further comprises cholesterol. In some embodiments, said lipid phase may comprise from 10 to 40 mol. % cholesterol, preferably from 15-25 mol. %. In one embodiment, said lipid phase may comprise about 30 mol. % POPC, about 10 mol. % DOTAP, about 20 mol. % CHEMS and about 40 mol. % Chol, the total being 100 mol. %.

The examples below give further mixtures of amphoteric liposomes suitable for practising the invention. As the invention is not limited to the examples, an assay for identifying and testing other amphoteric liposomes is also described.

The active drugs of the present invention are nucleic acid based. As mentioned above, these are classified into nucleic acids that encode one or more specific sequences for proteins, polypeptides or RNAs and into oligonucleotides that can specifically down-regulate protein expression.

In some embodiments of the invention, therefore, the nucleic acid based therapeutic may comprise a nucleic acid that is capable of being transcribed in a vertebrate cell into one or more RNAs, which RNAs may be mRNAs, shRNAs, miRNAs or ribozymes, wherein such mRNAs code for or more proteins or polypeptides. Such nucleic acid therapeutics may be circular DNA plasmids, linear DNA constructs, like MIDGE vectors (Minimalistic Immunogenically Defined Gene Expression) as disclosed in WO 98/21322 or DE 19753182, or mRNAs ready for translation (e.g. EP 1392341).

In another embodiment of the invention, oligonucleotides may be used that can target existing intracellular nucleic acids coding for a specific protein, thereby attenuating the expression of the protein. The term “target nucleic acid” encompasses DNA encoding a specific protein, as well as all RNAs derived from such DNA, being pre-mRNA or mRNA. A specific hybridisation between the target nucleic acid and one or more oligonucleotides directed against such sequences may result in an inhibition of protein expression. To achieve such specific targeting, the oligonucleotide should suitably comprise a continuous stretch of nucleotides that is complementary to the sequence of the target nucleic acid.

Oligonucleotides fulfilling the abovementioned criteria may comprehend a number of different chemistries or topologies. Oligonucleotides may be single stranded or double stranded. Single stranded oligonucleotides include, but are not limited to, DNA-based oligonucleotides, locked nucleic acids, 2′-modified oligonucleotides and others, commonly known as antisense oligonucleotides. Backbone or base modifications may include but are not limited to phosphothioate DNA (PTO), 2′ O-methyl RNA (2′Ome), 2′ O-methoxyethyl-RNA (2′MOE), peptide nucleic acids (PNA), N3′-P5′ phosphoamidates (NP), 2′fluoroarabino nucleic acids (FANA), locked nucleic acids (LNA), morpholine phosphoamidate (Morpholino), cyclohexene nucleic acid (CeNA), tricyclo-DNA (tcDNA) and others. Moreover, mixed chemistries are known in the art, being constructed from more than a single nucleotide species as copolymers, block-copolymers or gapmers or in other arrangements.

In addition to the aforementioned oligonucleotides, protein expression may also be inhibited using double stranded RNA molecules containing the complementary sequence motifs. Such RNA molecules are known as siRNA molecules in the art (e.g. WO 99/32619 and WO 02/055693). Again, various chemistries were adapted to this class of oligonucleotides. Also, DNA/RNA hybrid systems are known in the art.

In another embodiment of the present invention, decoy oligonucleotides may be used. These double stranded DNA molecules do not target nucleic acids, but transcription factors. This means that decoy oligonucleotides are adapted to bind sequence-specific DNA-binding proteins and interfere with the transcription (eg. Cho-Chung et al. in Curr Opin Mol Ther., 1999).

All above mentioned oligonucleotides may vary in length between as little as 10, preferably 15, and more preferably 18, and 50, preferably 30, and more preferably 25, nucleotides. The fit between the oligonucleotide and the target sequence is preferably perfect with each base of the oligonucleotide forming a base pair with its complementary base on the target nucleic acid over a continuous stretch of the abovementioned number of oligonucleotides. The pair of sequences may however contain one or a few mismatches within the said continuous stretch of base pairs, although this is less preferred.

The therapeutic agent may be selected according to the disease state or disorder to be treated or prevented. In some embodiments, the composition of the invention may comprise an oligonucleotide that targets nucleic acids encoding CD40, thereby to attenuate the expression of such CD40 in mammalian cells. As described above, by “nucleic acids encoding CD40” is meant herein DNA coding for CD40, as well as RNAs derived from such DNA, being pre-mRNA or mRNA.

In addition to the aforementioned oligonucleotides, CD40 expression may also be inhibited using double stranded RNA molecules containing complementary sequence motifs. Such RNA molecules are known in the art as siRNA molecules. Again, various chemistries are adapted to this class of oligonucleotides. Further, DNA/RNA hybrid systems are known in the art.

More specifically, reference is made here to U.S. Pat. No. 6,197,584 and US 2004/0186071, both to Bennett, which describe useful sequences and chemistries of such oligonucleotides. Reference is also made to Pluvinet, et al. in Blood, 2004, describing siRNA sequence motifs for the inhibition of CD40. Further siRNA motifs are in public domain and can be obtained, e.g. from Santa Cruz Biotechnology (Santa Cruz, U.S.A.).

Methods for the manufacturing of liposomes are known to those skilled in the art. They include extrusion through membranes of defined pore size, injection of lipid solutions in ethanol into the water phase containing cargo and high pressure homogenisation.

Also, it is known in the art that nucleic acid therapeutics can be contacted with an excipient at a substantially neutral pH, resulting in volume inclusion of a certain percentage of the solution containing the nucleic acid. High concentrations of excipients ranging from 50 mM to 150 mM are preferred to promote substantial encapsulation of the drug.

In contrast to such standard procedure, amphoteric liposomes offer the distinct advantage of binding nucleic acids at or below their isoelectric point and thereby concentrating the drug at the liposome surface. Such process is described in WO 02/066012, incorporated herein by reference, in more detail.

Irrespective of the actual production process any non-encapsulated active drug may be removed from the liposomes after the initial production step in which the liposomes are formed as tight containers. Again, the technical literature and the references included here describe such methodology in detail and suitable process steps may include but are not limited to size exclusion chromatography, sedimentation, dialysis, ultrafiltration or diafiltration and the like.

In preferred embodiments of the invention, at least 50 wt. % and preferably more than 80 wt. % of the drug is disposed inside the liposome.

However, such removal of non-encapsulated material is not mandatory, and in some embodiments of the invention, the composition may comprise free drug as well as entrapped drug.

The particle size of the composition may be between 50 and 1000 nm, preferably between 100 and 500 nm

After the manufacturing process, lyophilisation of the composition may provides a further means for stabilisation. In one preferred embodiment of the present invention, the composition may be lyophilized at the abovementioned acidic pH and then reconstituted with water for injection prior to use. The acidic pH during lyophilisation and subsequent reconstitution prevent loss of encapsulated nucleic acid material owing to an interaction of the drugs with the liposomal membrane. If lyophilisation is part of the manufacturing procedure, protecting agents such as sugars or amino acids or polymers may be present in the vehicle.

Although the application of the pharmaceutical composition is done with particular advantage at a lower pH, practising the invention is of course not limited to that. In some embodiments of the present invention, the composition may be applied at a physiological pH of between about 7 and about 8.

In one preferred embodiment of the present invention, the composition may be applied at a slightly acidic pH, in particular at a pH below the isoelectric point of the excipient. More preferably, the pH of the composition may be not lower than about pH 3.5, and most preferably the composition has a pH between 4 and 5 when applied. Pharmaceutically acceptable vehicles for such application are known to those skilled in the art and include, but are not limited to acetic acid, citric acid or glycine and the like for compositions having the desired pH. More generally, the vehicle may comprise any suitable pharmaceutically acceptable carrier comprising water, buffer substances, salts, sugars, polymers and the like.

As low pH may be detrimental to the long-term stability of the nucleic acid or lipids, the pH is preferentially adjusted to the lower value before use. Means to achieve this under pharmacologically acceptable standards are known to those of ordinary skill in the art and include, but are not limited to, mixing the storage stable colloid with an appropriate amount of acetic acid, citric acid or glycine, preferentially buffered to a lower pH, more preferred buffer between pH 2 and pH 4.

Following are particular combinations of process steps that may be used advantageously for preparing pharmaceutical compositions according to different embodiments of the present invention:
(A)

I. encapsulation of the nucleic acid at neutral pH

II. vehicle may be water, saline or buffered saline

III. actual liposome formation and sizing step

IV. non-entrapped drug removed

V. storage form: suspension

VI. pH is adjusted below the isoelectric point of the excipient

VII. administration at acidic pH
(B)

I. encapsulation of nucleic acid at neutral pH

II. vehicle may be water, saline or buffered saline

III. actual liposome formation and sizing step

IV. non-entrapped drug removed

V. pH is adjusted below the isoelectric point of the liposome excipient with the addition of protectants

VI. lyophilisation

VII. storage form: powder

VIII. reconstitution and administration at acidic pH
(C)

I. encapsulation of the nucleic acid at a pH below the isoelectric point of excipient using a molar ratio of cationic charges of the excipient to anionic charges of the drug between 0.5 and 20, preferably between 1 and 10
II. vehicle may be buffered with acetic acid, citric acid or the like and may further contain sodium chloride or sucrose.

III. actual liposome formation and sizing step

IV. addition of cryoprotectants and lyophilisation

V. storage form: powder

VI. reconstitution and administration at acidic pH
(D)

I. encapsulation of nucleic acid at a pH below the isoelectric point of the excipient using a molar ratio of cationic charges of the excipient to anionic charges of the drug between 0.5 and 20 and more preferred between 1 and 10

II. vehicle may be buffered with acetic acid, citric acid or the like and may further contain sodium chloride or sucrose.

III. actual liposome formation and sizing step

IV. raise pH to neutrality

V. non-entrapped drug removed

VI. select a combination of further process steps from (A), (B), (C).

The present invention thus comprehends a pharmaceutical composition comprising a nucleic acid for local application to a mucous membranes, ex vivo to a graft prior to transplantation or to the eye. Without being limited to the examples given here, such compositions may be therapeutically active in the treatment of inflammatory bowel disease. In general, the compositions of the invention are useful for the prevention or treatment of different conditions or diseases in mammals. One specific task is the local application of the compositions in the prevention or treatment of inflammations, immune or autoimmune disorders, including graft rejection, graft-versus-host disease, inflammatory bowel disease, Morbus Crohn, Colitis ulcerosa, Asthma bronchiale and COPD.

Administration of the composition of the invention is within the ordinary skill of those skilled in the art. Dosing may be dependent upon the severity and/or responsiveness of the disease to be treated, with the course of treatment lasting from several days to several months, or until a cure is effected or a diminution of the symptoms of the disease is achieved. Optimal dosing schedules can be calculated from measurements of drug accumulation in the body of the patient. Those of ordinary skill in the art can readily determine optimum dosages, dosing methodologies and repetition rates. Optimum dosages may vary depending on the relative potency of the individual drug in the composition and can generally be estimated based on EC50 values found to be effective in animal models. The dosage may be given daily, weekly, monthly or yearly or even less regularly. Those of ordinary skill in the art can easily estimate repetition rates for dosing based upon measured residence times and concentrations of the drug in body fluids or tissues.

Following successful treatment, it may be desirable to have the patient undergo maintenance therapy to prevent recurrence of the disease, wherein the formulation may be administered at maintenance doses, once or more daily to once per year.

### EXAMPLES

Following is a description by way of example only with reference to the accompanying drawings of embodiments of the present invention.

### Example 1

Preparation of Amphoteric Liposomes

TABLE 1

| Lipids | Composition |
|---|---|
| POPC/DOTAP/CHEMS | 60:10:30 |
| POPC/DOTAP/CHEMS | 40:15:45 |
| POPC/DOTAP/CHEMS | 20:20:60 |
| POPC/DOTAP/CHEMS | 25:75 |

A mixture of lipids was dissolved in chloroform and evaporated in a round bottom flask to dryness under vacuum. Lipid films were hydrated with PBS, pH 7.5. The resulting lipid concentration was 50 mM. The suspensions were hydrated for 25 minutes in a water bath at room temperature, sonicated for 5 minutes and frozen at −70° C. After thawing the liposomal suspensions were extruded 15 times through polycarbonate membranes with a pore size of 200nm.

### Example 2

pH-Shift Experiment With Empty Amphoteric Liposomes

10 μl liposomes of Example 1 were diluted 1:100 in 100 mM Citrate/Phosphate-buffer pH 4-8 and incubated for one hour at room temperature. Then 7.5 ml 0.9% saline was added and the size of the liposomes was characterized by dynamic light scattering.

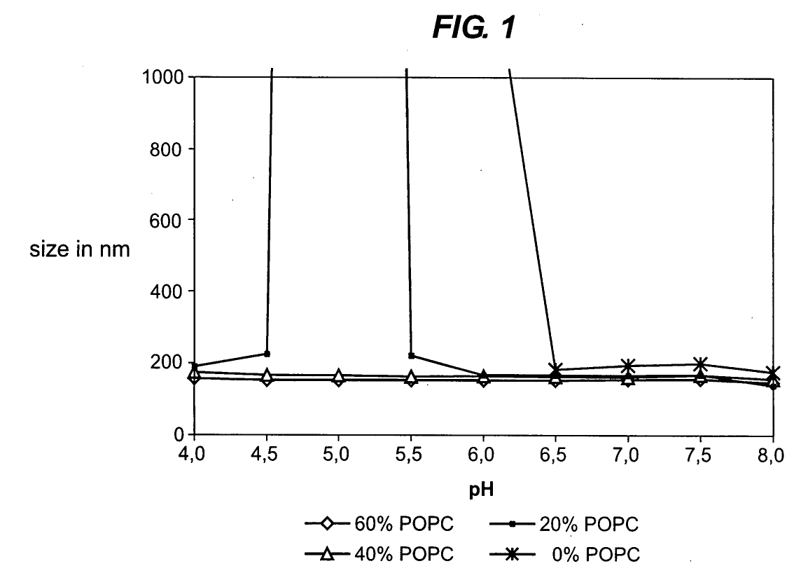

Results are presented in FIG. 1. Amphoteric liposomes built up of the charged lipids DOTAP and CHEMS in a ratio 1:3 are only stable at an acidic pH when the neutral lipid POPC is also present in the bilayer with at least 40%.

### Example 3

Preparation of Carboxyfluorescein (CF) Loaded Liposomes

TABLE 2

| Lipids | Composition |
|---|---|
| POPC/DOTAP/CHEMS | 20:40:40 |
| POPC/DOTAP/CHEMS | 20:30:50 |
| POPC/DOTAP/CHEMS | 20:20:60 |
| POPC/DOTAP/CHEMS | 20:10:70 |
| POPC/DOTAP/CHEMS | 20:0:80 |

TABLE 3

| Lipids | Composition |
|---|---|
| DOPE/DOTAP/CHEMS | 20:40:40 |
| DOPE/DOTAP/CHEMS | 20:30:50 |
| DOPE/DOTAP/CHEMS | 20:20:60 |
| DOPE/DOTAP/CHEMS | 20:10:70 |
| DOPE/DOTAP/CHEMS | 20:0:80 |

A mixture of lipids was dissolved in chloroform and evaporated in a round bottom flask to dryness under vacuum. Lipid films were hydrated with 10 μM CF in 10 mM Hepes, 150 mM NaCl, pH 7.5. The resulting lipid concentration was 10 mM. The suspensions were hydrated for 45 minutes in a water bath at room temperature, sonicated for 5 minutes following by three freeze/thaw cycles at −70° C. After thawing the liposomal suspensions were extruded 15 times through polycarbonate membranes with a pore size of 200 nm. Non-encapsulated CF was removed by size exclusion chromatography, whereas the liposomes were diluted six fold.

### Example 4

pH-Shift Experiment With Amphoteric Liposomes of Example 3

A mixture of 150 μl liposomes of example 3, 7.5 ml 0.9% saline and 150 μl 0.5M Citrate/Phosphate-buffer pH 4-8 was prepared and the size of the liposomes was characterized by dynamic light scattering.

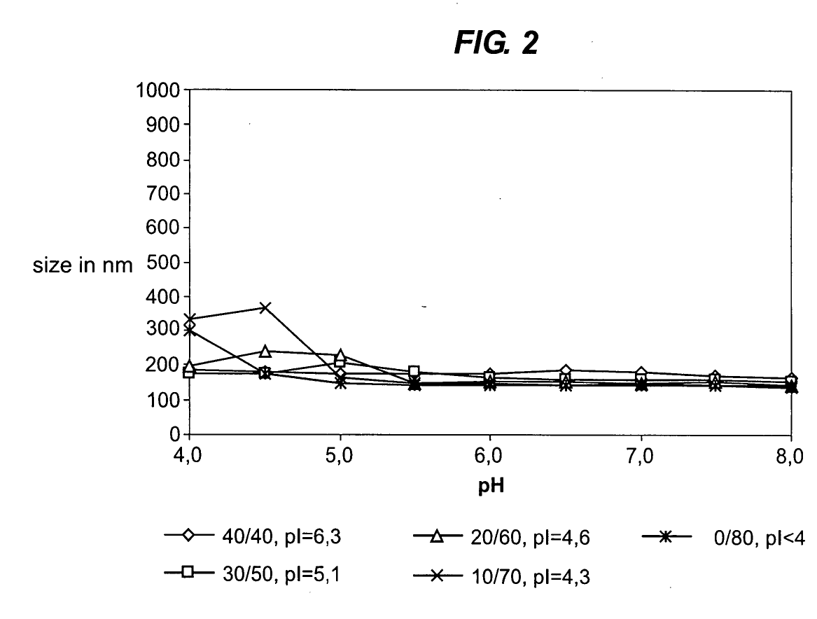

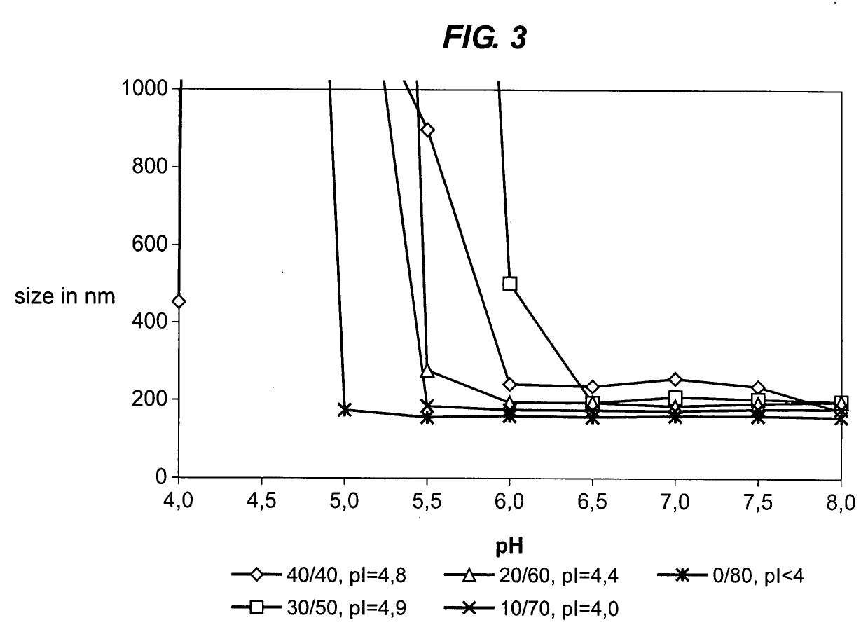

Results are presented in FIGS. 2 and 3. Amphoteric liposomes built up of the charged lipids DOTAP and CHEMS in different ratios can be stabilized by the presence of POPC but not with DOPE.

### Example 5

Preparation of Empty Amphoteric Liposomes

TABLE 4

| Lipids | Composition |
|---|---|
| POPC/DOPE/MoChol/CHEMS | 15:45:20:20 |
| POPC/DOTAP/CHEMS/Chol | 30:10:20:40 |
| POPC/DOTAP/CHEMS | 60:10:30 |
| POPC/DOTAP/CHEMS | 60:20:20 |
| POPC/DOPE/MoChol/DMG- | 6:24:46:23 |
| Succ | |

A mixture of lipids was dissolved in chloroform and evaporated in a round bottom flask to dryness under vacuum. Lipid films were hydrated with PBS, pH 7.5. The resulting lipid concentration was 100 mM. The suspensions were hydrated for 25 minutes in a water bath at room temperature, sonicated for 5 minutes and frozen at −70° C. After thawing the liposomal suspensions were extruded 15 times through polycarbonate membranes with a pore size of 400 nm.

### Example 6

Preparation of Plasmid-Loaded Amphoteric Liposomes

TABLE 5

| Lipids | Composition | Plasmid |
|---|---|---|
| POPC/DOPE/MoChol/CHEMS | 15:45:20:20 | inside + outside |
| POPC/DOTAP/CHEMS | 60:10:30 | Inside |
| POPC/MoChol/CHEMS | 30:35:35 | Inside |
| | | inside + outside |

Liposomes were produced by injecting 10 Vol-% of an ethanolic lipid solution into 10 mM NaAc 150 mM NaCl pH 4.5 or 10 mM NaAc pH 4.5 containing 16 μg/ml of a 7000 bp plasmid encoding for luciferase. The resulting lipid concentration was 2 mM. The pH of this solution was immediately shifted with 1/10 volume 1M Hepes pH 8. To concentrate the diluted liposomes the suspensions were sedimented for 1 h at 80.000 rpm in a TLA 100.4 rotor (Beckman Optima-MAX). To remove non-encapsulated plasmid the concentrated liposomal suspensions were diluted with a sucrose stock solution and brought to 0.8M sucrose. 0.5M sucrose in PBS and pure PBS were layered on top, forming a gradient for removing the plasmid outside of the particles. Sucrose gradients were spun for 45 min at 40.000 rpm in a MLS-50 rotor (Beckman Optima-MAX) and the liposomes were taken from the upper interphase.

The formulation POPC/DOTAP/CHEMS60:10:30 was manufactured by following process:

The lipid mixture was dissolved in chloroform and evaporated in a round bottom flask to dryness under vacuum. Lipid films were hydrated with 10 mM NaAc/150 mM NaCl, pH4.5 containing 100 μg/ml plasmid PBS. The resulting lipid concentration was 10 mM. The suspensions were hydrated for 25 minutes in a water bath at room temperature, sonicated for 5 minutes and frozen at −70° C. After thawing the liposomal suspensions were extruded 15 times through polycarbonate membranes with a pore size of 800/200/800 nm. To remove non-encapsulated plasmid the concentrated liposomal suspensions were diluted with a sucrose stock solution and brought to 0.8M sucrose. 0.5M sucrose in PBS and pure PBS were layered on top, forming a gradient for removing the plasmid outside of the particles. Sucrose gradients were spun for 45 min at 40.000 rpm in a MLS-50 rotor (Beckman Optima-MAX) and the liposomes were taken from the upper interphase.

### Example 7

Stable Amphoteric Liposomes at pH 4.5

Liposomes were first diluted 1:10 in PBS pH 7.5 and afterwards 1/10 Vol 1M Acetate, pH 4.5 was added very fast. The samples were vortexed immediately after the addition of the shift buffer. Liposomes were characterized by dynamic light scattering.

TABLE 6
stable amphoteric liposomes after pH-Shift to pH 4.5

| | | Size/PI | Size/PI |
|---|---|---|---|
| Formulation | Cargo | pH 7.5 | pH 4.5 |
| POPC/DOPE/MoChol/CHEMS | plasmid | 117/0.373 | 266/0.244 |
| 15:45:20:20 | in + out | | |
| | Empty | 193/0.255 | 212/0.195 |
| POPC/DOTAP/CHEMS/Chol | Empty | 190/0.208 | 202/0.218 |
| 30:10:20:40 | | | |
| POPC/DOTAP/CHEMS | plasmid | 125/0.091 | 145/0.296 |
| 60:10:30 | inside | | |
| | Empty | 180/0.053 | 179/0.08 |
| POPC/DOTAP/CHEMS | Empty | 169/0.138 | 164/0.101 |
| 60:20:20 | | | |
| POPC/MoChol/CHEMS | plasmid | 109/0.479 | 154/0.240 |
| 30:35:35 | in + out | | |
| | plasmid | 190/0.177 | 234/0.283 |
| | inside | | |
| POPC/DOPE/MoChol/DMG- | empty | 217/0.113 | 240/0.200 |
| Succ | | | |
| 6:24:46:23 | | | |

### Example 8

Preparation of CD40-ODN-Containing Liposomes

A mixture of 85 μmol POPC, 42 μmol CHEMS and 14 μmol DOTAP was dissolved in chloroform and evaporated in a round bottom flask to dryness under vacuum.

ODN with the sequence T*C*C*TAGATGGACCGCT*G*T was used with asterisks indicating a phosphorothioate linkage between the nucleotides (after Gao, Ph.D. thesis, Goettingen 2003, rAS3).

Lipid films were hydrated with 1 mg ODN in 1 mL of buffer (10 mM sodium acetate, 150 mM NaCl pH 4.5). The suspensions were hydrated for 25 minutes in a water bath at room temperature, sonicated for 5 minutes and eventually frozen at −70° C. After thawing the liposomal suspensions were extruded 15 times through polycarbonate membranes with a pore size of 400 nm. The liposome suspensions were brought to pH 7.5 using 1M HEPES buffer and to 0.8M sucrose using a stock solution. Non-encapsulated ODN was removed from the extruded sample by flotation through 0.5M sucrose overlaid with 10 mM HEPES, 150 mM NaCl pH 7.5 and the liposome suspension was stored at 4° C. Resulting liposomes were characterized by dynamic light scattering and found to be 220 to 250 mn in size.

### Example 9

Colitis Induction

Colitis was induced by using a single intra-colonic application of 2,4,6-trinitrobenzene sulphonic acid (TNBS) prepared by adding 20 mg of TNBS to 135 μl of 35% ethanol in 150 mM NaCl. Male Wistar rats (200 . . . 250 g) were placed under light ether anaesthesia and the mixture was administered using an 8 cm long catheter inserted through the anal canal into the descending colon. After removing the catheter, rats were held in a headfirst position for 30 s to avoid flowing out of the enema and rats were kept under normal condition afterwards.

### Example 10

Treatment and Analysis

Rats were treated with CD40 antisense from example 1 either 4 hours before or 3 days after the colitis induction. The antisense suspension from Example 1 was brought to pH 4.5 using 1M buffered acetic acid/sodium acetate pH 4.0 and a total of 100 μl containing 2.7 μg CD40 antisense suspension was applied to the colon according to Example 2.

Seven days after induction of the colitis the animals were sacrificed. The colon was removed and opened longitudinally. Colon samples were fixed in PBS containing 4% formaldehyde. Paraffin-embedded sections (5 μm) were stained with haematoxylin/eosin followed by microscopic inspection.

Colonic damage was scored according to the following criteria:

TABLE 1
Criteria for microscopic scoring of colonic damage.

| Parameters | Score |
|---|---|
| Ulceration | |
| No | 0 |
| Minor | 1 |
| Major | 2 |
| Inflammation | |
| None | 0 |
| Minor | 1 |
| Major | 2 |
| Severe | 3 |
| Depth of lesion | |
| None | 0 |
| Superficial | 1 |
| One third | 2 |
| Two third | 3 |
| Transmural | 4 |
| Fibrosis | |
| None | 0 |
| Minor | 1 |
| Major | 2 |
| Lymphocyte infiltration | |
| No | 0 |
| Yes | 1 |
| Total score | 0-12 |

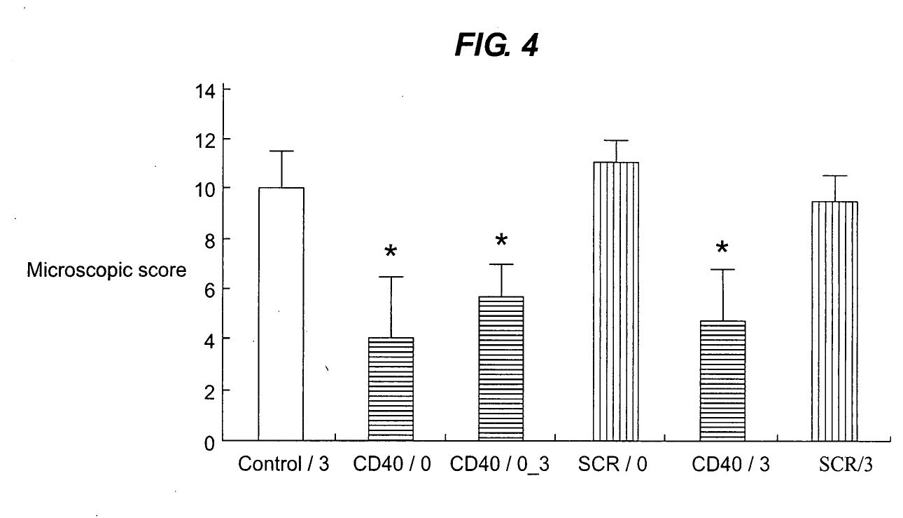

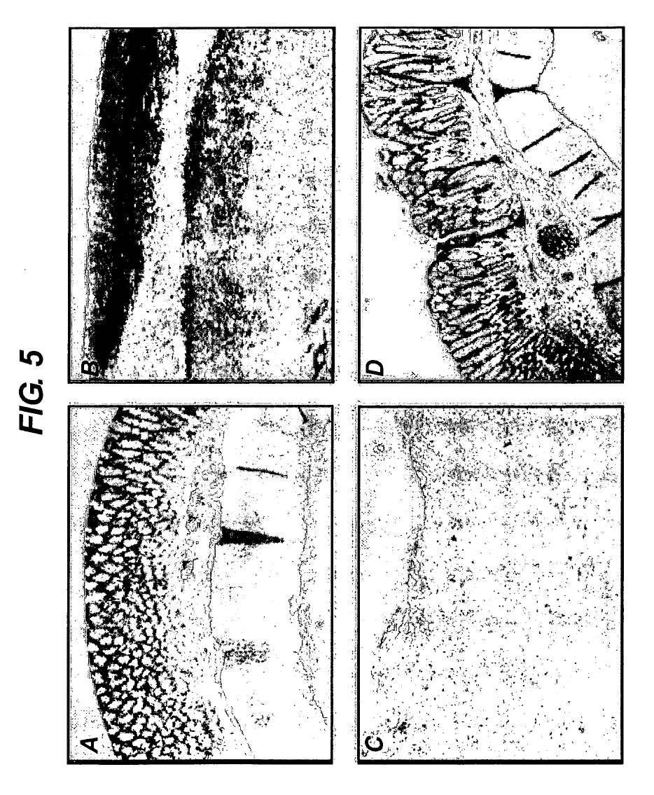

Results are presented in the FIGS. 4 to 5A-5D and demonstrate a very substantial reduction of the experimental colitis when treated with antisense directed against CD40, but not with the scrambled control antisense. Quite surprisingly, even a single treatment of a fully developed colitis at day 3 resulted in a strong and almost complete reduction of the inflammation. In confirmation to that, prevention of the colitis was also achieved when the formulation was applied in a preventive mode before the initiation of the disease.

### Example 11

Alternative Formulation

When used as excipient, a mixture of 60 mol. % POPC, 20 mol. % HistChol and 20 mol. % Cholesterol also resulted in successful treatment of the experimental colitis.

### Example 12

Non Removal of Outside Antisense

When used as a formulation, non-removal of non encapsulated antisense also resulted in carrier systems that are stable colloids.

### Example 13

Materials

This example provides non-limiting examples of CD40 nucleotide sequences that may be targeted by oligonucleotides that modulate the expression of CD40 and that are suitable for use in the compositions in accordance with the present invention.

Human CD40 mRNA (GenBank accession no. X60592)

Human CD40 mRNA sequence for targeting in accordance with the present invention is presented in SEQ ID NO:1. Related sequence information is found in published patent application number US 2004/0186071 (i.e., SEQ ID NO:85) to Bennett, et al. and in U.S. Pat. No. 6,197,584 (i.e., SEQ ID NO:85) to Bennett, et al. and in Pluvinet, et al., Blood, 2004, 104(12), 3642-3646, the contents of which are incorporated by reference herein.

(SEQ ID NO:1):

| | | | | |
|---|---|---|---|---|
| 1 | gcctcgctcg | ggcgcccagt | ggtcctgccg | cctggtctca |
| | cctcgccatg | gttcgtctgc | | |
| 61 | ctctgcagtg | cgtcctctgg | ggctgcttgc | tgaccgctgt |
| | ccatccagaa | ccacccactg | | |
| 121 | catgcagaga | aaaacagtac | ctaataaaca | gtcagtgctg |
| | ttctttgtgc | cagccaggac | | |
| 181 | agaaactggt | gagtgactgc | acagagttca | ctgaaacgga |
| | atgccttcct | tgcggtgaaa | | |
| 241 | gcgaattcct | agacacctgg | aacagagaga | cacactgcca |
| | ccagcacaaa | tactgcgacc | | |
| 301 | ccaacctagg | gcttcgggtc | cagcagaagg | gcacctcaga |
| | aacagacacc | atctgcacct | | |
| 361 | gtgaagaagg | ctggcactgt | acgagtgagg | cctgtgagag |
| | ctgtgtcctg | caccgctcat | | |
| 421 | gctcgcccgg | ctttggggtc | aagcagattg | ctacaggggt |
| | ttctgatacc | atctgcgagc | | |
| 481 | cctgcccagt | cggcttcttc | tccaatgtgt | catctgcttt |
| | cgaaaaatgt | cacccttgga | | |
| 541 | caagctgtga | gaccaaagac | ctggttgtgc | aacaggcagg |
| | cacaaacaag | actgatgttg | | |
| 601 | tctgtggtcc | ccaggatcgg | ctgagagccc | tggtggtgat |
| | ccccatcatc | ttcgggatcc | | |
| 661 | tgtttgccat | cctcttggtg | ctggtcttta | tcaaaaaggt |
| | ggccaagaag | ccaaccaata | | |
| 721 | aggcccccca | ccccaagcag | gaaccccagg | agatcaattt |
| | tcccgacgat | cttcctggct | | |
| 781 | ccaacactgc | tgctccagtg | caggagactt | tacatggatg |
| | ccaaccggtc | acccaggagg | | |
| 841 | atggcaaaga | gagtcgcatc | tcagtgcagg | agagacagtg |
| | aggctgcacc | cacccaggag | | |
| 901 | tgtggccacg | tgggcaaaca | ggcagttggc | cagagagcct |
| | ggtgctgctg | ctgcaggggt | | |
| 961 | gcaggcagaa | gcggggagct | atgcccagtc | |
| | agtgccagcc | cctc | | |

Mus musculus CD40 mRNA

Murine CD40 mRNA sequence for targeting in accordance with the present invention is presented in SEQ ID NO:2. Related sequence information is found in published patent application number US 2004/0186071 (i.e. SEQ ID NO:132) to Bennett, et al., the contents of which are incorporated by reference herein.

(SEQ ID NO:2):

| | | | | |
|---|---|---|---|---|
| gcctcctggc | ccttcagctg | tggtctttcc | cgttttctga | 60 |
| ctttgcggtg | acactgggga | | | |
| cttccttaga | cctctctgga | gacgctttcg | gttctgcaga | 120 |
| gattcccagg | ggtattgtgg | | | |
| gtggggtggg | gtaacaatag | tgtccctgtg | gcgctcccag | 180 |
| tccctatagt | aatccttcac | | | |
| ccctctgcta | tcttgcaatc | aggagagtcc | ttagccctgc | 240 |
| tataggtggc | ttttgaggtc | | | |
| ctggatgcga | ggagggggac | tggggggtgg | gtcgggtaat | 300 |
| gtaagaaaag | ggctcctttt | | | |
| gggaccctgg | ctcctccagc | caccttggtg | cccatccctt | 360 |
| aaactcttgg | ggacaatcag | | | |
| actcctggga | aggtcctggg | gaaatccctg | ctcagtgact | 420 |
| agccataggc | ccaccgcgat | | | |
| tggtgcccga | agaccccgcc | ctcttcctgg | gcgggactcc | 480 |
| tagcagggac | tttggagtga | | | |
| cttgtggctt | cagcaggagc | cctgtgattt | ggctcttctg | 540 |
| atctcgccct | gcgatggtgt | | | |
| ctttgcctcg | gctgtgcgcg | ctatggggct | gcttgttgac | 600 |
| agcggtgagt | ggcttgtgtt | | | |
| ctaacctcca | agggagttag | ggcttagaga | gtgagagatg | 660 |
| gaaagaggaa | agaggagaca | | | |
| agactttgga | gatgagagat | cttcctactg | gaagcggcgg | 720 |
| ttagtaggat | gggcaagatc | | | |
| tctcgcgtct | tgacacacac | acacacacac | acaaatgagg | 780 |
| tgggctgctc | ctctttcctt | | | |
| ccagaaggtc | ggggttctgt | tccacgaagc | ccacagggaa | 840 |
| ccttagggag | ggcattcctc | | | |
| cacagcggtg | cctggacagc | tttgtctgac | ccaagccttg | 900 |
| ctccggagct | gactgcagag | | | |
| actggaaagg | gttagcagac | aggaagcctg | gctggggg | 938 |

Rat CD40 mRNA (GenBank Accession No. AF 241231)

Rat CD40 mRNA sequence for targeting in accordance with the present invention is presented in SEQ ID NO:3. (See, Gao, Ph.D. thesis, Goettingen 2003).

(SEQ ID NO:3):

| | | | | |
|---|---|---|---|---|
| 1 | tgggacccct | gtgatctggc | tgctctgatc | tcgctctgca |
| | atgctgcctt | tgcctcagct | | |
| 61 | gtgcgcgctc | tggggctgct | tgttgacagc | ggtccatcta |
| | ggacagtgtg | ttacgtgcag | | |
| 121 | tgacaaacag | tacctccaag | gtggcgagtg | ctgcgatttg |
| | tgccagccgg | gaaaccgact | | |
| 181 | agttagccac | tgcacagctc | ttgagaagac | ccaatgccaa |
| | ccgtgcgact | caggcgaatt | | |
| 241 | ctcagctcac | tggaacaggg | agatccgctg | ccaccagcac |
| | cgacactgcg | aactcaatca | | |
| 301 | agggcttcag | gttaagaagg | agggcaccgc | ggtntcagac |
| | actgtttgta | cctgcaagga | | |
| 361 | agggcagcac | tgcgccagca | aggagtgcga | gacgtgcgct |
| | cagcacaggc | cctgtggccc | | |
| 421 | tggctttgga | gtcgtgcaga | tggccactga | gactactgat |
| | accgtctgcc | aaccctgccc | | |
| 481 | ggtcggattc | ttctccaatg | ggtcatcact | ttttgaaaag |
| | tgtcatccat | ggacaagctg | | |
| 541 | tgaagat | | | |

Porcine CD40 cDNA

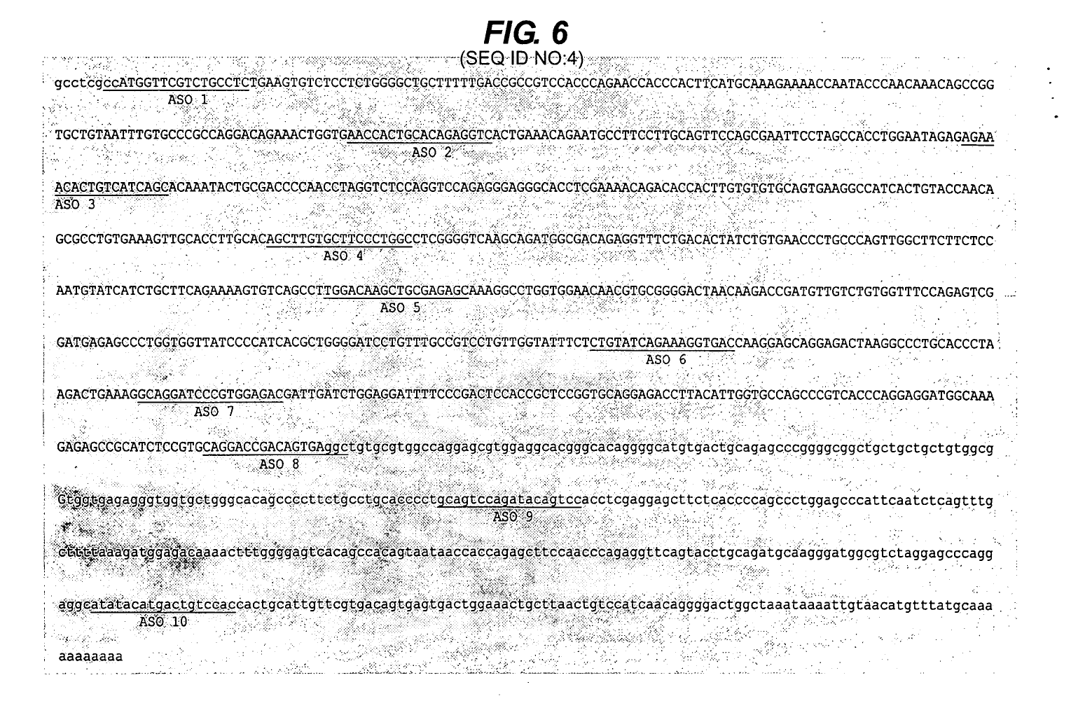

Porcine CD40 cDNA sequence for targeting in accordance with the present invention is presented in SEQ ID NO:4. (FIG. 6). Related sequence information is found in Rushworth, et al., Transplantation, 2002, 73(4), 635-642, the contents of which are incorporated by reference herein.

In addition, the following provide non-limiting examples of anti-CD40 oligonucleotides, e.g., antisense CD40 nucleic acid sequences, that are suitable for use in the present invention:

Oligonucleotides Against Human CD40

Examples of human antisense CD40 oligonucleotides are presented below. Further sequence information is found in published patent application number US 2004/0186071 and U.S. Pat. No. 6,197,584 to Bennett, et al., the contents of which are provided by reference herein. The SEQ ID NOs referred to by Bennett, et al. are provided to the right.

| | | | |
|---|---|---|---|
| SEQ ID NO: 5 | ccaggcggca ggaccact | Seq ID No: 1 | of Bennett et al. |
| SEQ ID NO: 6 | gaccaggcgg caggacca | Seq ID No.: 2 | of Bennett et al. |
| SEQ ID NO: 7 | aggtgagacc aggcggca | Seq ID No: 3 | of Bennett et al. |
| SEQ ID NO: 8 | gcagaggcag acgaacca | Seq ID No: 5 | of Bennett et al. |
| SEQ ID NO: 9 | gcaagcagcc ccagagga | Seq ID No: 6 | of Bennett et al. |
| SEQ ID NO: 10 | ggtcagcaag cagcccca | Seq ID No.: 7 | of Bennett et al. |
| SEQ ID NO: 11 | gacagcggtc agcaagca | Seq ID No: 8 | of Bennett et al. |
| SEQ ID NO: 12 | gatggacagc ggtcagca | Seq ID No: 9 | of Bennett et al. |
| SEQ ID NO: 13 | tctggatgga cagcggtc | Seq ID No.: 10 | of Bennett et al. |
| SEQ ID NO: 14 | ggtggttctg gatggaca | Seq ID No: 11 | of Bennett et al. |
| SEQ ID NO: 15 | gtgggtggtt ctggatgg | Seq ID No: 12 | of Bennett et al. |
| SEQ ID NO: 16 | gcagtgggtg gttctgga | Seq ID No: 13 | of Bennett et al. |
| SEQ ID NO: 17 | ctggcacaaa gaacagca | Seq ID No: 15 | of Bennett et al. |
| SEQ ID NO: 18 | gtgcagtcac tcaccagt | Seq ID No: 20 | of Bennett et al. |
| SEQ ID NO: 19 | attccgtttc agtgaact | Seq ID No: 23 | of Bennett et al. |
| SEQ ID NO: 20 | ttcaccgcaa ggaaggca | Seq ID No: 25 | of Bennett et al. |
| SEQ ID NO: 21 | ctctgttcca ggtgtcta | Seq ID No: 26 | of Bennett et al. |
| SEQ ID NO: 22 | ctggtggcag tgtgtctc | Seq ID No: 27 | of Bennett et al. |
| SEQ ID NO: 23 | ggtgcccttc tgctggac | Seq ID No: 31 | of Bennett et al. |
| SEQ ID NO: 24 | ctgaggtgcc cttctgct | Seq ID No: 32 | of Bennett et al. |
| SEQ ID NO: 25 | gtgtctgttt ctgaggtg | Seq ID No: 33 | of Bennett et al. |
| SEQ ID NO: 26 | acaggtgcag atggtgtc | Seq ID No: 35 | of Bennett et al. |
| SEQ ID NO: 27 | gtgccagcct tcttcaca | Seq ID No: 37 | of Bennett et al. |
| SEQ ID NO: 28 | tgcaggacac agctctca | Seq ID No: 40 | of Bennett et al. |
| SEQ ID NO: 29 | gagcggtgca ggacacag | Seq ID No: 41 | of Bennett et al. |
| SEQ ID NO: 30 | aatctgcttg accccaaa | Seq ID No: 43 | of Bennett et al. |
| SEQ ID NO: 31 | gctcgcagat ggtatcag | Seq ID No: 46 | of Bennett et al. |
| SEQ ID NO: 32 | gcagggctcg cagatggt | Seq ID No: 47 | of Bennett et al. |
| SEQ ID NO: 33 | gactgggcag ggctcgca | Seq ID No: 49 | of Bennett et al. |
| SEQ ID NO: 34 | gcagatgaca cattggag | Seq ID No: 52 | of Bennett et al. |
| SEQ ID NO: 35 | tcgaaagcag atgacaca | Seq ID No: 53 | of Bennett et al. |
| SEQ ID NO: 36 | gtccaagggt gacatttt | Seq ID No: 54 | of Bennett et al. |
| SEQ ID NO: 37 | caggtctttg gtctcaca | Seq ID No: 57 | of Bennett et al. |
| SEQ ID NO: 38 | ctgttgcaca accaggtc | Seq ID No: 58 | of Bennett et al. |
| SEQ ID NO: 39 | gtttgtgcct gcctgttg | Seq ID No: 59 | of Bennett et al. |
| SEQ ID NO: 40 | gtcttgtttg tgcctgcc | Seq ID No: 60 | of Bennett et al. |
| SEQ ID NO: 41 | caccaccagg gctctcag | Seq ID No: 64 | of Bennett et al. |
| SEQ ID NO: 42 | gggatcacca ccagggct | Seq ID No: 65 | of Bennett et al. |
| SEQ ID NO: 43 | gtcgggaaaa ttgatctc | Seq ID No: 71 | of Bennett et al. |
| SEQ ID NO: 44 | ggagccagga agatcgtc | Seq ID No: 73 | of Bennett et al. |
| SEQ ID NO: 45 | tggagccagg aagatcgt | Seq ID No: 74 | of Bennett et al. |
| SEQ ID NO: 46 | tggcatccat gtaaagtc | Seq ID No: 77 | of Bennett et al. |
| SEQ ID NO: 47 | ggtgcagcct cactgtct | Seq ID No: 81 | of Bennett et al. |
| SEQ ID NO: 48 | aactgcctgt ttgcccac | Seq ID No: 82 | of Bennett et al. |

The following siRNA sequences are suitable for use in the present invention. (See, e.g., Pluvinet, et al., Blood, 2004, 104(12), 3642-3646), the contents of which are incorporated by reference herein.

| | |
|---|---|
| (SEQ ID NO:49): | |
| 5_-GCGAAUUCCUAGACACCUGUU-3_{—} | (siRNA-2 of Pluvinet et al.) |
| 3_-UUCGCUUAAGGAUCUGUGGAC-5_{—} | |
| (SEQ ID NO:50): | |
| 5_-CUGGUGAGUGACUGCACAGUU-3_{—} | (siRNA-6 of Pluvinet et al.) |
| 3_-UUGACCACUCACUGACGUGUC-5_{—} | |
| (SEQ ID NO:51): | |
| 5_-UACUGCGACCCCAACCUAGUU-3_{—} | (siRNA-8 of Pluvinet et al.) |
| 3_-UUAUGACGCUGGGGUUGGAUC-5_{—} | |

All siRNA contain a 2 nucleotide overhang at 3′ ends.

Oligonucleotides Against Murine CD40

Examples of murine antisense CD40 oligonucleotides are presented below. Further sequence information is found in published patent application number US 2004/0186071 to Bennett, et al., the contents of which are hereby incorporated by reference herein. The SEQ ID NOs referred to by Bennett, et al. are provided to the right.

Murine

| | | | |
|---|---|---|---|
| SEQ ID NO: 52 | agacaccatc gcag | Seq. ID No. 116 | of Bennett et al. |
| SEQ ID NO: 53 | gcgagatcag aagag | Seq. ID No. 117 | of Bennett et al. |
| SEQ ID NO: 54 | cgctgtcaac aagca | Seq. ID No. 118 | of Bennett et al. |
| SEQ ID NO: 55 | ctgccctaga tggac | Seq. ID No. 119 | of Bennett et al. |
| SEQ ID NO: 56 | ctggctggca caaat | Seq. ID No. 120 | of Bennett et al. |
| SEQ ID NO: 57 | cttgtccagg gataa | Seq. ID No. 123 | of Bennett et al. |
| SEQ ID NO: 58 | cacagatgac attag | Seq. ID No. 124 | of Bennett et al. |
| SEQ ID NO: 59 | tgatatagag aaaca | Seq. ID No. 125 | of Bennett et al. |
| SEQ ID NO: 60 | ctcattatcc tttgg | Seq. ID No. 127 | of Bennett et al. |
| SEQ ID NO: 61 | ggttcagacc agg | Seq. ID No. 128 | of Bennett et al. |
| SEQ ID NO: 62 | tttatttagc cagta | Seq. ID No. 130 | of Bennett et al. |
| SEQ ID NO: 63 | agccccacgc actgg | Seq. ID No. 131 | of Bennett et al. |
| SEQ ID NO: 64 | tctcactcct atcccagt | Seq. ID No. 134 | of Bennett et al. |
| SEQ ID NO: 65 | attagtctga ctcgt | Seq. ID No. 138 | of Bennett et al. |
| SEQ ID NO: 66 | acattagtct gactc | Seq. ID No. 139 | of Bennett et al. |
| SEQ ID NO: 67 | cagatgacat tagtc | Seq. ID No. 142 | of Bennett et al. |
| SEQ ID NO: 68 | ctggactcac cacag | Seq. ID No. 143 | of Bennett et al. |
| SEQ ID NO: 69 | ggactcacca cagat | Seq. ID No. 144 | of Bennett et al. |
| SEQ ID NO: 70 | actcaccaca gatga | Seq. ID No. 145 | of Bennett et al. |
| SEQ ID NO: 71 | tcaccacaga tgaca | Seq. ID No. 146 | of Bennett et al. |
| SEQ ID NO: 72 | accacagatg acatt | Seq. ID No. 147 | of Bennett et al. |
| SEQ ID NO: 73 | agatgacatt ag | Seq. ID No. 153 | of Bennett et al. |
| SEQ ID NO: 74 | cagatgacat tag | Seq. ID No. 154 | of Bennett et al. |
| SEQ ID NO: 75 | acagatgaca ttag | Seq. ID No. 155 | of Bennett et al. |
| SEQ ID NO: 76 | ccacagatga cattag | Seq. ID No. 156 | of Bennett et al. |
| SEQ ID NO: 77 | accacagatg acattag | Seq. ID No. 157 | of Bennett et al. |
| SEQ ID NO: 78 | caccacagat gacattag | Seq. ID No. 158 | of Bennett et al. |
| SEQ ID NO: 79 | tcaccacaga tgacattag | Seq. ID No. 159 | of Bennett et al. |
| SEQ ID NO: 80 | ctcaccacag atgacattag | Seq. ID No. 160 | of Bennett et al. |

Oligonucleotides Against Rat CD40

Examples of rat antisense CD40 oligonucleotides are presented below. (See, Gao, Ph.D. thesis, 2003, University of Göttingen, Germany).

| | | |
|---|---|---|
| SEQ ID NO:81 | accgctgtcaacaagcagc | (rAS2 of Gao) |
| SEQ ID NO:82 | tcctagatggaccgctgt | (rAS3 of Gao) |
| SEQ ID NO:83 | taacacactgtcctag | (rAS4 of Gao) |

Oligonucleotides Against Porcine CD40

Examples of porcine antisense CD40 oligonucleotides are presented below. See, Rushworth, et al., Transplantation, 2002, 73(4), 635-642, the contents of which are incorporated by reference herein.

SEQ ID NO:84

| | |
|---|---|
| gctgatgacagtgtttct | (Aso3 of Rushworth et al.) |

SEQ ID NO:85

| | |
|---|---|
| gcctcactctcgctcctg | (Aso8 of Rushworth et al.) |

SEQ ID NO:86

| | |
|---|---|
| ggactgtatctggactgc | (Aso9 of Rushworth et al.) |

SEQ ID NO:87

| | |
|---|---|
| gtggacagtcatgtatat | (Aso10 of Rushworth et al.) |

Glossary of Abbreviated Lipid Names

Abbreviations for lipids refer primarily to standard use in the literature and are included here as a helpful reference:

| | |
|---|---|
| DMPC | Dimyristoylphosphatidylcholine |
| DPPC | Dipalmitoylphosphatidylcholine |
| DSPC | Distearoylphosphatidylcholine |
| POPC | Palmitoyl-oleoylphosphatidylcholine |
| DOPC | Dioleoylphosphatidylcholine |
| DOPE | Dioleoylphosphatidylethanolamine |
| DMPE | Dimyristoylphosphatidylethanolamine |
| DPPE | Dipalmitoylphosphatidylethanolamine |
| DOPG | Dioleoylphosphatidylglycerol |
| POPG | Palmitoyl-oleoylphosphatidylglycerol |
| DMPG | Dimyristoylphosphatidylglycerol |
| DPPG | Dipalmitoylphosphatidylglycerol |
| DMPS | Dimyristoylphosphatidylserine |
| DPPS | Dipalmitoylphosphatidylserine |
| DOPS | Dioleoylphosphatidylserine |
| POPS | Palmitoyl-oleoylphosphatidylserine |
| DMPA | Dimyristoylphosphatidic acid |
| DPPA | Dipalmitoylphosphatidic acid |
| DOPA | Dioleoylphosphatidic acid |
| POPA | Palmitoyl-oleoylphosphatidic acid |
| CHEMS | Cholesterolhemisuccinate |
| DC-Chol | 3-β-[N-(N′,N′-dimethylethane) carbamoyl]cholesterol |
| CetylP | Cetylphosphate |
| DODAP | (1,2)-dioleoyloxypropyl)-N,N-dimethylammonium chloride |
| DOEPC | 1,2-dioleoyl-sn-glycero-3-ethylphosphocholine |
| DAC-Chol | 3-β-[N-(N,N′-dimethylethane) carbamoyl]cholesterol |
| TC-Chol | 3-β-[N-(N′,N′,N′-trimethylaminoethane) carbamoyl] cholesterol |
| DOTMA | (1,2-dioleyloxypropyl)-N,N,N-trimethylammonium chloride) (Lipofectin ®) |
| DOGS | ((C18)₂GlySper3⁺) N,N-dioctadecylamido-glycyl-spermin (Transfectam ®) |
| CTAB | Cetyl-trimethylammoniumbromide, |
| CPyC | Cetyl-pyridiniumchloride |
| DOTAP | (1,2-dioleoyloxypropyl)-N,N,N-trimethylammonium salt |
| DMTAP | (1,2-dimyristoyloxypropyl)-N,N,N-trimethylammonium salt |
| DPTAP | (1,2-dipalmitoyloxypropyl)-N,N,N-trimethylammonium salt |
| DOTMA | (1,2-dioleyloxypropyl)-N,N,N-trimethylammonium chloride) |
| DORIE | (1,2-dioleyloxypropyl)-3 dimethylhydroxyethyl ammoniumbromide) |
| DDAB | Dimethyldioctadecylammonium bromide |
| DPIM | 4-(2,3-bis-palmitoyloxy-propyl)-1-methyl-1H-imidazole |
| CHIM | Cholesterol-(3-imidazol-1-yl propyl)carbamate |
| MoChol | 4-(2-Aminoethyl)-Morpholino-Cholesterolhemisuccinate |
| HisChol | Histaminyl-Cholesterolhemisuccinate. |
| HCChol | Nα-Histidinyl-Cholesterolcarbamate |
| HistChol | Nα-Histidinyl-Cholesterol-hemisuccinate. |
| AC | Acylcarnosine, Stearyl-& Palmitoylcarnosine |
| HistDG | 1,2-Dipalmitoylglycerol-hemisuccinate-Nα-Histidinyl-hemisuccinate, & Distearoyl-, Dimyristoyl, Dioleoyl or |
| | palmitoyl-oleoylderivatives |
| IsoHistSuccDG | 1,2-Dipalmitoylglycerol-Oα-Histidinyl-Nα-hemisuccinat, & Distearoyl-, Dimyristoyl, Dioleoyl or |
| | palmitoyl-oleoylderivatives |
| DGSucc | 1,2-Dipalmitoyglycerol-3-hemisuccinate & Distearoyl-, dimyristoyl-Dioleoyl or palmitoyl-oleoylderivatives |

MoChol

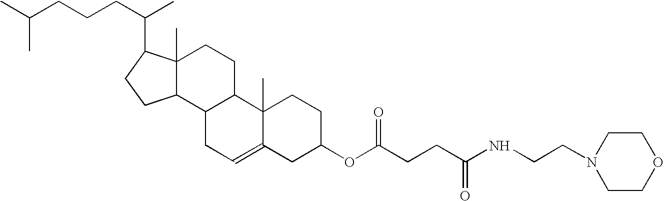

DG-Succ

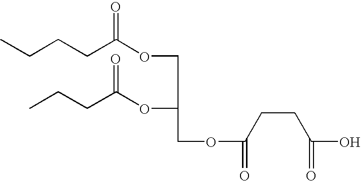

DOTAP

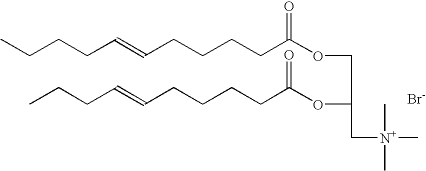

IsohistsuccDG

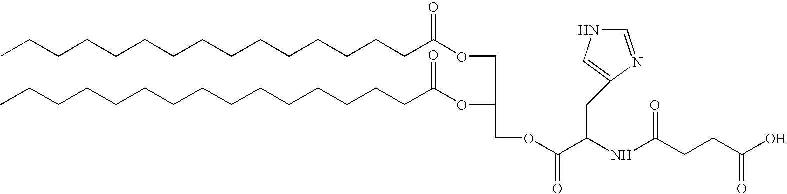

HisChol

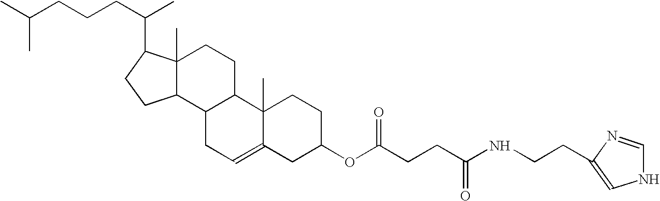

HCChol

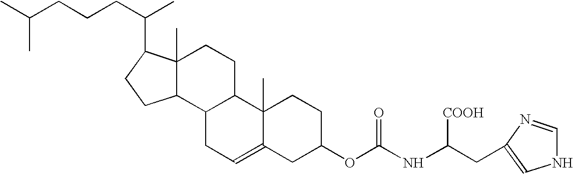

AC

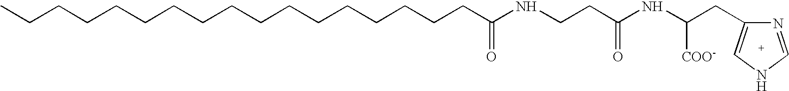

Hist-Chol

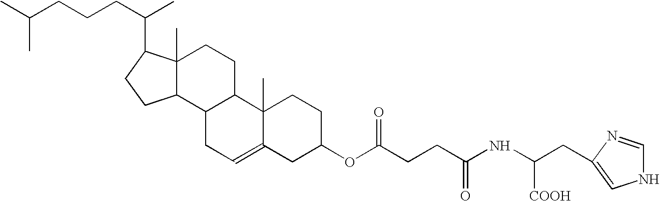

Hist-DG

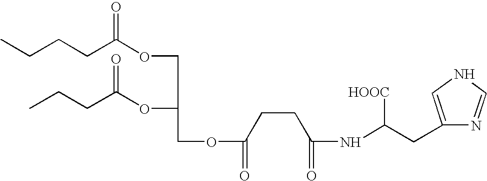

What is claimed is:

1. A pharmaceutical composition comprising a nucleic acid as a therapeutic agent, an excipient and a pharmaceutically acceptable vehicle therefor, said excipient comprising an amphoteric liposome having an isoelectric point between 4 and 7.4, wherein said composition is formulated to have a pH in the range 3 to 5.

2. The pharmaceutical composition according to claim 1, wherein said composition is formulated to have a pH in the range 4 to 5.

3. The pharmaceutical composition according to claim 1, wherein said amphoteric liposome is formed from a lipid phase comprising an amphoteric lipid, or a mixture of lipid components with amphoteric properties, and a neutral phospholipid.

4. The pharmaceutical composition according to claim 3, wherein said neutral phospholipid includes a phosphatidylcholine.

5. The pharmaceutical composition according to claim 4, wherein said phosphatidylcholine is selected from the group consisting of POPC, natural or hydrogenated soy bean PC, natural or hydrogenated egg PC, DMPC, DPPC or DOPC.

6. The pharmaceutical composition according to claim 4, wherein said phosphatidylcholine comprises POPC, non-hydrogenated soy bean PC, or non-hydrogenated egg PC.

7. The pharmaceutical composition according to claim 4, wherein said neutral phospholipid comprises a mixture of a phosphatidylcholine and a phosphatidylethanolamine.

8. The pharmaceutical composition according to claim 7, wherein said phosphatidylethanolamine is selected from the group consisting of DOPE or DMPE and DPPE.

9. The pharmaceutical composition according to claim 7, wherein said phosphatidylcholine comprises POPC, soy PC or egg PC, and said phosphatidylethanolamine comprises DOPE.

10. The pharmaceutical composition according to claim 4, wherein said neutral phospholipid constitutes at least 20 mol. % of said lipid phase.

11. The pharmaceutical composition according to claim 3, wherein said amphoteric lipid comprises a single lipid that is selected from the group consisting of HistChol, HistDG, isoHistSuccDG, Acylcamosin and HCChol.

12. The pharmaceutical composition according to claim 11, wherein said amphoteric lipid is HistChol.

13. The pharmaceutical composition according to claim 3, wherein said lipid components with amphoteric properties comprise a mixture of two or more anionic and cationic lipids, said cationic lipid or lipids being selected from the group consisting of DMTAP, DPTAP, DOTAP, DC-Chol, MoChol, HisChol, DPIM, CHIM, DORIE, DDAB, DAC-Chol, TC-Chol, DOTMA, DOGS, (C18)₂Gly⁺ N,N-dioctadecylamido-glycine, CTAP, CPyC, DODAP and DOEPC, and said anionic lipid or lipids being selected from the group consisting of DGSucc, DMPS, DPPS, DOPS, POPS, DMPG, DPPG, DOPG, POPG, DMPA, DPPA, DOPA, POPA, CHEMS and CetylP.

14. The pharmaceutical composition according to claim 13, wherein said cationic lipids comprise one or more of DOTAP, DC-Chol, MoChol and HisChol,

15. The pharmaceutical composition according to claim 13, wherein said anionic lipids comprise one or more of DMGSucc, DOGSucc, DOPA, CHEMS and CetylP.

16. The pharmaceutical composition according to claim 13, wherein said lipid phase comprises POPC, DOTAP and CHEMS and comprises a greater molar amount of CHEMS than DOTAP.

17. The pharmaceutical composition according to claim 16, wherein said lipid phase comprises 20-60 mol. % POPC, 10-40 mol. % DOTAP and 20-70 mol. % CHEMS, the total being 100 mol. %.

18. The pharmaceutical composition according to claim 17, wherein said lipid phase comprises about 60 mol. % POPC, about 10 mol. % DOTAP and about 30 mol. % CHEMS, the total being 100 mol. %.

19. The pharmaceutical composition according to claim 13, wherein said lipid phase comprises POPC, MoChol and CHEMS.

20. The pharmaceutical composition according to claim 19, wherein MoChol is present in said lipid phase in a molar amount that is substantially equal to or exceeds the molar amount of CHEMS.

21. The pharmaceutical composition according to claim 20, wherein said lipid phase comprises about 30 mol. % POPC, about 35 mol. % MoChol and about 35 mol. % CHEMS, the total being 100 mol. %.

22. The pharmaceutical composition according to claim 19, wherein said lipid phase further comprises DOPE.

23. The pharmaceutical composition according to claim 22, wherein said lipid phase comprises MoChol in greater or substantially equal molar amounts to CHEMS, and CHEMS and MoCHOL are present in a molar amount between about 30 and about 80 mol. % of the lipid phase

24. The pharmaceutical composition according to claim 23, wherein said lipid phase comprises about 15 mol. % POPC, about 45 mol. % DOPE, about 20 mol. % MoChol and about 20 mol. % CHEMS, the total being 100 mol. %.

25. The pharmaceutical composition according to claim 23, wherein said lipid phase comprises about 6 mol. % POPC, about 24 mol. % DOPE, about 46 mol. % MoChol and about 23 mol. % CHEMS, the total being 100 mol. %

26. The pharmaceutical composition according to claim 13, wherein said lipid phase comprises POPC, DOPE, MoChol and DMGSucc.

27. The pharmaceutical composition according to claim 26, wherein said lipid phase comprises MoCHol in greater or substantially equal molar amounts to DMG-Succ, and the total molar amount of DMG-Succ and MoCHOL is between 30 and 80 mol. % of the lipid phase.

28. The pharmaceutical composition according to claim 27, wherein said lipid phase comprises about 15 mol. % POPC, about 45 mol. % DOPE, about 20 mol. % MoChol and about 20 mol. % DMG-Succ, the total being 100 mol. %.

29. The pharmaceutical composition according to claim 27, wherein said lipid phase comprises about 6 mol. % POPC, about 24 mol. % DOPE, about 46 mol. % MoChol and about 23 mol. % DMGSucc, the total being 100 mol. %.

30. The pharmaceutical composition according to claim 3, wherein said lipid phase further comprises cholesterol.

31. The pharmaceutical composition according to claim 30, wherein said lipid phase comprises about 30 mol. % POPC, about 10 mol. % DOTAP, about 20 mol. % CHEMS and about 40 mol. % Chol, the total being 100 mol. %.

32. The pharmaceutical composition according to claim 1, wherein said amphoteric liposome has a size in the range 50 to 1000 nm.

33. The pharmaceutical composition according to claim 1, wherein said nucleic acid acid is capable of being transcribed in a vertebrate cell into one or more RNAs, said RNAs being mRNAs, shRNAs, miRNAs or ribozymes, said mRNAs coding for one or more proteins or polypeptides.

34. The pharmaceutical composition according to claim 1, wherein said nucleic acid is a circular DNA plasmid, a linear DNA construct, or an mRNA.

35. The pharmaceutical composition according to claim 1, wherein said nucleic acid is an oligonucleotide.

36. The pharmaceutical composition according to claim 35, wherein said oligonucleotide is an antisense oligonucleotide of 15 to 50 basepairs in length.

37. The pharmaceutical composition according to claim 35, wherein said oligonucleotide contains phosphothioate linkages.

38. The pharmaceutical composition according to claim 35, wherein said oligonucleotide contains 2′MOE modified nucleobases.

39. The pharmaceutical composition according to claim 35, wherein said oligonucleotide contains LNA nucleobases or FANA nucleobases.

40. The pharmaceutical composition according to claim 35, wherein said oligonucleotide contains naturally occurring ribonucleotides or deoxyribonucleotides.

41. The pharmaceutical composition according to claim 35, wherein said oligonucleotide comprises a siRNA of 15 to 30 basepairs in length.

42. The pharmaceutical composition according to claim 35, wherein said oligonucleotide is a decoy oligonucleotide of 15 to 30 basepairs in length.

43. The pharmaceutical composition according to claim 1, wherein a portion of said nucleic acid is disposed within said liposome.

44. The pharmaceutical composition according to claim 43, wherein at least 50 mol. % of said nucleic acid is disposed within said liposome.

45. The pharmaceutical composition according to claim 43, wherein at least 80 mol. % of said nucleic acid is disposed within said liposome.

46. The pharmaceutical composition according to claim 1, wherein said composition includes non-encapsulated nucleic acids.

47. The pharmaceutical composition according to claim 1, wherein said composition is lyophilised at an acidic pH for subsequent reconstitution with essentially unbuffered water or saline.

48. The pharmaceutical composition according to claim 1, said composition applied locally to a mucous membrane, to a graft prior to transplantation, or to the eye.

49. The pharmaceutical composition according to claim 48, wherein said composition is applied locally to a mucous membrane in the nose, airways, mouth, intestine or vagina.

50. A method of treatment or prophylaxis of an inflammatory, immune, or autoimmune condition or disorder, comprising: administering to a human or non-human animal patient in need thereof a pharmaceutically or prophylacticly effective amount of the pharmaceutical composition according to claim 1.

51. A method of treating a graft prior to transplantation, comprising: administering to said graft ex vivo the pharmaceutical composition according to claim 1.

52. A method of vaccinating a human or non-human animal with a genetic vaccine, comprising: administering to said human or animal an effective amount of the pharmaceutical composition according to claim 1.

53. The method according to claim 50, wherein said composition is acidified at the time of use to a pH in the range 3 to 5.

54. The method according to claim 51, wherein said composition is acidified at the time of use to a pH in the range 3 to 5.

55. The method according to claim 52, wherein said composition is acidified at the time of use to a pH in the range 3 to 5.

56. A kit comprising a pharmaceutical composition and instructions for use thereof, said composition comprising a nucleic acid as a therapeutic agent, an excipient comprising an amphoteric liposome and a pharmaceutically acceptable vehicle therefor, wherein said amphoteric liposome has an isoelectric point between 4 and 7.4, and said composition is provided in the form of a suspension at substantially neutral pH, said instructions directing the acidification of said suspension to a pH in the range of about 3 to about 5 prior to use.

57. The kit according to claim 56, further comprising separate acidifying means for admixture to the suspension at the time of use for buffering said composition to said lower pH.

58. The kit according to claim 57, wherein said acidifying means comprises acetic acid, citric acid or glycine.

59. A kit comprising a pharmaceutical composition and instructions for the use thereof, said composition comprising a nucleic acid as a therapeutic agent, an excipient comprising an amphoteric liposome, and a pharmaceutically acceptable vehicle therefor, wherein said liposome has an isoelectric point of between 4 and 7.4 and wherein said composition is provided in lyophilised form such that upon reconstitution with an aqueous medium, the pH of the reconstituted composition is in the range of about 3 to about 5, said instructions directing the reconstitution of the lyophilised composition at the time of use.

60. The kit according to claim 59, further comprising a separate aqueous medium for reconstitution of said composition at the time of use.

61. The kit according to claim 60, wherein said aqueous medium comprises substantially unbuffered water of saline.


SEQUENCE LISTING (91 sequences)

SEQ ID NO: 1 - DNA, 1004 bp, Homo sapiens
gcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgcaggggtgcaggcagaagcggggagctatgcccagtcagtgccagcccctc

SEQ ID NO: 2 - DNA, 938 bp, Mus musculus
gcctcctggcccttcagctgtggtctttcccgttttctgactttgcggtgacactggggacttccttagacctctctggagacgctttcggttctgcagagattcccaggggtattgtgggtggggtggggtaacaatagtgtccctgtggcgctcccagtccctatagtaatccttcacccctctgctatcttgcaatcaggagagtccttagccctgctataggtggcttttgaggtcctggatgcgaggagggggactggggggtgggtcgggtaatgtaagaaaagggctccttttgggaccctggctcctccagccaccttggtgcccatcccttaaactcttggggacaatcagactcctgggaaggtcctggggaaatccctgctcagtgactagccataggcccaccgcgattggtgcccgaagaccccgccctcttcctgggcgggactcctagcagggactttggagtgacttgtggcttcagcaggagccctgtgatttggctcttctgatctcgccctgcgatggtgtctttgcctcggctgtgcgcgctatggggctgcttgttgacagcggtgagtggcttgtgttctaacctccaagggagttagggcttagagagtgagagatggaaagaggaaagaggagacaagactttggagatgagagatcttcctactggaagcggcggttagtaggatgggcaagatctctcgcgtcttgacacacacacacacacacacaaatgaggtgggctgctcctctttccttccagaaggtcggggttctgttccacgaagcccacagggaaccttagggagggcattcctccacagcggtgcctggacagctttgtctgacccaagccttgctccggagctgactgcagagactggaaagggttagcagacaggaagcctggctggggg

SEQ ID NO: 3 - DNA, 547 bp, Rattus sp.
  modified_base (334): a, c, g, t, unknown or other
tgggacccctgtgatctggctgctctgatctcgctctgcaatgctgcctttgcctcagctgtgcgcgctctggggctgcttgttgacagcggtccatctaggacagtgtgttacgtgcagtgacaaacagtacctccaaggtggcgagtgctgcgatttgtgccagccgggaaaccgactagttagccactgcacagctcttgagaagacccaatgccaaccgtgcgactcaggcgaattctcagctcactggaacagggagatccgctgccaccagcaccgacactgcgaactcaatcaagggcttcaggttaagaaggagggcaccgcggtntcagacactgtttgtacctgcaaggaagggcagcactgcgccagcaaggagtgcgagacgtgcgctcagcacaggccctgtggccctggctttggagtcgtgcagatggccactgagactactgataccgtctgccaaccctgcccggtcggattcttctccaatgggtcatcactttttgaaaagtgtcatccatggacaagctgtgaagat

SEQ ID NO: 4 - DNA, 1284 bp, Sus sp.
gcctcgccatggttcgtctgcctctgaagtgtctcctctggggctgctttttgaccgccgtccacccagaaccacccacttcatgcaaagaaaaccaatacccaacaaacagccggtgctgtaatttgtgcccgccaggacagaaactggtgaaccactgcacagaggtcactgaaacagaatgccttccttgcagttccagcgaattcctagccacctggaatagagagaaacactgtcatcagcacaaatactgcgaccccaacctaggtctccaggtccagagggagggcacctcgaaaacagacaccacttgtgtgtgcagtgaaggccatcactgtaccaacagcgcctgtgaaagttgcaccttgcacagcttgtgcttccctggcctcggggtcaagcagatggcgacagaggtttctgacactatctgtgaaccctgcccagttggcttcttctccaatgtatcatctgcttcagaaaagtgtcagccttggacaagctgcgagagcaaaggcctggtggaacaacgtgcggggactaacaagaccgatgttgtctgtggtttccagagtcggatgagagccctggtggttatccccatcacgctggggatcctgtttgccgtcctgttggtatttctctgtatcagaaaggtgaccaaggagcaggagactaaggccctgcaccctaagactgaaaggcaggatcccgtggagacgattgatctggaggattttcccgactccaccgctccggtgcaggagaccttacattggtgccagcccgtcacccaggaggatggcaaagagagccgcatctccgtgcaggagcgagagtgaggctgtgcgtggccaggagcgtggaggcacgggcacaggggcatgtgactggagagcccggggcggctgctgctgctgtggcggtggtgagagggtggtgctgggcacagccccttctgcctgcacccctgcagtccagatacagtccacctcgaggagcttctcaccccagccctggagcccattcaatctcagtttgcttttaaagatggagacaaaactttggggagtcacagccacagtaataaccaccagagcttccaacccagaggttcagtacctgcagatgcaagggatggcgtctaggagcccaggaggcatatacatgactgtccaccactgcattgttcgtgacagtgagtgactggaaactgcttaactgtccatcaacaggggactggctaaataaaattgtaacatgtttatgcaaaaaaaaaaa

SEQ ID NO: 5 - DNA, 18 bp, Artificial Sequence
  Description of Artificial Sequence Synthetic oligonucleotide
ccaggcggcaggaccact

SEQ ID NO: 6 - DNA, 18 bp, Artificial Sequence
  Description of Artificial Sequence Synthetic oligonucleotide
gaccaggcggcaggacca

SEQ ID NO: 7 - DNA, 18 bp, Artificial Sequence
  Description of Artificial Sequence Synthetic oligonucleotide
aggtgagaccaggcggca

SEQ ID NO: 8 - DNA, 18 bp, Artificial Sequence
  Description of Artificial Sequence Synthetic oligonucleotide
gcagaggcagacgaacca

SEQ ID NO: 9 - DNA, 18 bp, Artificial Sequence
  Description of Artificial Sequence Synthetic oligonucleotide
gcaagcagccccagagga

SEQ ID NO: 10 - DNA, 18 bp, Artificial Sequence
  Description of Artificial Sequence Synthetic oligonucleotide
ggtcagcaagcagcccca

SEQ ID NO: 11 - DNA, 18 bp, Artificial Sequence
  Description of Artificial Sequence Synthetic oligonucleotide
gacagcggtcagcaagca

SEQ ID NO: 12 - DNA, 18 bp, Artificial Sequence
  Description of Artificial Sequence Synthetic oligonucleotide
gatggacagcggtcagca

SEQ ID NO: 13 - DNA, 18 bp, Artificial Sequence
  Description of Artificial Sequence Synthetic oligonucleotide
tctggatggacagcggtc

SEQ ID NO: 14 - DNA, 18 bp, Artificial Sequence
  Description of Artificial Sequence Synthetic oligonucleotide
ggtggttctggatggaca

SEQ ID NO: 15 - DNA, 18 bp, Artificial Sequence
  Description of Artificial Sequence Synthetic oligonucleotide
gtgggtggttctggatgg

SEQ ID NO: 16 - DNA, 18 bp, Artificial Sequence
  Description of Artificial Sequence Synthetic oligonucleotide
gcagtgggtggttctgga

SEQ ID NO: 17 - DNA, 18 bp, Artificial Sequence
  Description of Artificial Sequence Synthetic oligonucleotide
ctggcacaaagaacagca

SEQ ID NO: 18 - DNA, 18 bp, Artificial Sequence
  Description of Artificial Sequence Synthetic oligonucleotide
gtgcagtcactcaccagt

SEQ ID NO: 19 - DNA, 18 bp, Artificial Sequence
  Description of Artificial Sequence Synthetic oligonucleotide
attccgtttcagtgaact

SEQ ID NO: 20 - DNA, 18 bp, Artificial Sequence
  Description of Artificial Sequence Synthetic oligonucleotide
ttcaccgcaaggaaggca

SEQ ID NO: 21 - DNA, 18 bp, Artificial Sequence
  Description of Artificial Sequence Synthetic oligonucleotide
ctctgttccaggtgtcta

SEQ ID NO: 22 - DNA, 18 bp, Artificial Sequence
  Description of Artificial Sequence Synthetic oligonucleotide
ctggtggcagtgtgtctc

SEQ ID NO: 23 - DNA, 18 bp, Artificial Sequence
  Description of Artificial Sequence Synthetic oligonucleotide
ggtgcccttctgctggac

SEQ ID NO: 24 - DNA, 18 bp, Artificial Sequence
  Description of Artificial Sequence Synthetic oligonucleotide
ctgaggtgcccttctgct

SEQ ID NO: 25 - DNA, 18 bp, Artificial Sequence
  Description of Artificial Sequence Synthetic oligonucleotide
gtgtctgtttctgaggtg

SEQ ID NO: 26 - DNA, 18 bp, Artificial Sequence
  Description of Artificial Sequence Synthetic oligonucleotide
acaggtgcagatggtgtc

SEQ ID NO: 27 - DNA, 18 bp, Artificial Sequence
  Description of Artificial Sequence Synthetic oligonucleotide
gtgccagccttcttcaca

SEQ ID NO: 28 - DNA, 18 bp, Artificial Sequence
  Description of Artificial Sequence Synthetic oligonucleotide
tgcaggacacagctctca

SEQ ID NO: 29 - DNA, 18 bp, Artificial Sequence
  Description of Artificial Sequence Synthetic oligonucleotide
gagcggtgcaggacacag

SEQ ID NO: 30 - DNA, 18 bp, Artificial Sequence
  Description of Artificial Sequence Synthetic oligonucleotide
aatctgcttgaccccaaa

SEQ ID NO: 31 - DNA, 18 bp, Artificial Sequence
  Description of Artificial Sequence Synthetic oligonucleotide
gctcgcagatggtatcag

SEQ ID NO: 32 - DNA, 18 bp, Artificial Sequence
  Description of Artificial Sequence Synthetic oligonucleotide
gcagggctcgcagatggt

SEQ ID NO: 33 - DNA, 18 bp, Artificial Sequence
  Description of Artificial Sequence Synthetic oligonucleotide
gactgggcagggctcgca

SEQ ID NO: 34 - DNA, 18 bp, Artificial Sequence
  Description of Artificial Sequence Synthetic oligonucleotide
gcagatgacacattggag

SEQ ID NO: 35 - DNA, 18 bp, Artificial Sequence
  Description of Artificial Sequence Synthetic oligonucleotide
tcgaaagcagatgacaca

SEQ ID NO: 36 - DNA, 18 bp, Artificial Sequence
  Description of Artificial Sequence Synthetic oligonucleotide
gtccaagggtgacatttt

SEQ ID NO: 37 - DNA, 18 bp, Artificial Sequence
  Description of Artificial Sequence Synthetic oligonucleotide
caggtctttggtctcaca

SEQ ID NO: 38 - DNA, 18 bp, Artificial Sequence
  Description of Artificial Sequence Synthetic oligonucleotide
ctgttgcacaaccaggtc

SEQ ID NO: 39 - DNA, 18 bp, Artificial Sequence
  Description of Artificial Sequence Synthetic oligonucleotide
gtttgtgcctgcctgttg

SEQ ID NO: 40 - DNA, 18 bp, Artificial Sequence
  Description of Artificial Sequence Synthetic oligonucleotide
gtcttgtttgtgcctgcc

SEQ ID NO: 41 - DNA, 18 bp, Artificial Sequence
  Description of Artificial Sequence Synthetic oligonucleotide
caccaccagggctctcag

SEQ ID NO: 42 - DNA, 18 bp, Artificial Sequence
  Description of Artificial Sequence Synthetic oligonucleotide
gggatcaccaccagggct

SEQ ID NO: 43 - DNA, 18 bp, Artificial Sequence
  Description of Artificial Sequence Synthetic oligonucleotide
gtcgggaaaattgatctc

SEQ ID NO: 44 - DNA, 18 bp, Artificial Sequence
  Description of Artificial Sequence Synthetic oligonucleotide
ggagccaggaagatcgtc

SEQ ID NO: 45 - DNA, 18 bp, Artificial Sequence
  Description of Artificial Sequence Synthetic oligonucleotide
tggagccaggaagatcgt

SEQ ID NO: 46 - DNA, 18 bp, Artificial Sequence
  Description of Artificial Sequence Synthetic oligonucleotide
tggcatccatgtaaagtc

SEQ ID NO: 47 - DNA, 18 bp, Artificial Sequence
  Description of Artificial Sequence Synthetic oligonucleotide
ggtgcagcctcactgtct

SEQ ID NO: 48 - DNA, 18 bp, Artificial Sequence
  Description of Artificial Sequence Synthetic oligonucleotide
aactgcctgtttgcccac

SEQ ID NO: 49 - RNA, 21 nt, Artificial Sequence
  Description of Artificial Sequence Synthetic siRNA sequence
gcgaauuccuagacaccuguu

SEQ ID NO: 50 - RNA, 21 nt, Artificial Sequence
  Description of Artificial Sequence Synthetic siRNA sequence
cuggugagugacugcacaguu

SEQ ID NO: 51 - RNA, 21 nt, Artificial Sequence
  Description of Artificial Sequence Synthetic siRNA sequence
uacugcgaccccaaccuaguu

SEQ ID NO: 52 - DNA, 14 bp, Artificial Sequence
  Description of Artificial Sequence Synthetic oligonucleotide
agacaccatcgcag

SEQ ID NO: 53 - DNA, 15 bp, Artificial Sequence
  Description of Artificial Sequence Synthetic oligonucleotide
gcgagatcagaagag

SEQ ID NO: 54 - DNA, 15 bp, Artificial Sequence
  Description of Artificial Sequence Synthetic oligonucleotide
cgctgtcaacaagca

SEQ ID NO: 55 - DNA, 15 bp, Artificial Sequence
  Description of Artificial Sequence Synthetic oligonucleotide
ctgccctagatggac

SEQ ID NO: 56 - DNA, 15 bp, Artificial Sequence
  Description of Artificial Sequence Synthetic oligonucleotide
ctggctggcacaaat

SEQ ID NO: 57 - DNA, 15 bp, Artificial Sequence
  Description of Artificial Sequence Synthetic oligonucleotide
cttgtccagggataa

SEQ ID NO: 58 - DNA, 15 bp, Artificial Sequence
  Description of Artificial Sequence Synthetic oligonucleotide
cacagatgacattag

SEQ ID NO: 59 - DNA, 15 bp, Artificial Sequence
  Description of Artificial Sequence Synthetic oligonucleotide
tgatatagagaaaca

SEQ ID NO: 60 - DNA, 15 bp, Artificial Sequence
  Description of Artificial Sequence Synthetic oligonucleotide
ctcattatcctttgg

SEQ ID NO: 61 - DNA, 13 bp, Artificial Sequence
  Description of Artificial Sequence Synthetic oligonucleotide
ggttcagaccagg

SEQ ID NO: 62 - DNA, 15 bp, Artificial Sequence
  Description of Artificial Sequence Synthetic oligonucleotide
tttatttagccagta

SEQ ID NO: 63 - DNA, 15 bp, Artificial Sequence
  Description of Artificial Sequence Synthetic oligonucleotide
agccccacgcactgg

SEQ ID NO: 64 - DNA, 18 bp, Artificial Sequence
  Description of Artificial Sequence Synthetic oligonucleotide
tctcactcctatcccagt

SEQ ID NO: 65 - DNA, 15 bp, Artificial Sequence
  Description of Artificial Sequence Synthetic oligonucleotide
attagtctgactcgt

SEQ ID NO: 66 - DNA, 15 bp, Artificial Sequence
  Description of Artificial Sequence Synthetic oligonucleotide
acattagtctgactc

SEQ ID NO: 67 - DNA, 15 bp, Artificial Sequence
  Description of Artificial Sequence Synthetic oligonucleotide
cagatgacattagtc

SEQ ID NO: 68 - DNA, 15 bp, Artificial Sequence
  Description of Artificial Sequence Synthetic oligonucleotide
ctggactcaccacag

SEQ ID NO: 69 - DNA, 15 bp, Artificial Sequence
  Description of Artificial Sequence Synthetic oligonucleotide
ggactcaccacagat

SEQ ID NO: 70 - DNA, 15 bp, Artificial Sequence
  Description of Artificial Sequence Synthetic oligonucleotide
actcaccacagatga

SEQ ID NO: 71 - DNA, 15 bp, Artificial Sequence
  Description of Artificial Sequence Synthetic oligonucleotide
tcaccacagatgaca

SEQ ID NO: 72 - DNA, 15 bp, Artificial Sequence
  Description of Artificial Sequence Synthetic oligonucleotide
accacagatgacatt

SEQ ID NO: 73 - DNA, 12 bp, Artificial Sequence
  Description of Artificial Sequence Synthetic oligonucleotide
agatgacattag

SEQ ID NO: 74 - DNA, 13 bp, Artificial Sequence
  Description of Artificial Sequence Synthetic oligonucleotide
cagatgacattag

SEQ ID NO: 75 - DNA, 14 bp, Artificial Sequence
  Description of Artificial Sequence Synthetic oligonucleotide
acagatgacattag

SEQ ID NO: 76 - DNA, 16 bp, Artificial Sequence
  Description of Artificial Sequence Synthetic oligonucleotide
ccacagatgacattag

SEQ ID NO: 77 - DNA, 17 bp, Artificial Sequence
  Description of Artificial Sequence Synthetic oligonucleotide
accacagatgacattag

SEQ ID NO: 78 - DNA, 18 bp, Artificial Sequence
  Description of Artificial Sequence Synthetic oligonucleotide
caccacagatgacattag

SEQ ID NO: 79 - DNA, 19 bp, Artificial Sequence
  Description of Artificial Sequence Synthetic oligonucleotide
tcaccacagatgacattag

SEQ ID NO: 80 - DNA, 20 bp, Artificial Sequence
  Description of Artificial Sequence Synthetic oligonucleotide
ctcaccacagatgacattag

SEQ ID NO: 81 - DNA, 19 bp, Artificial Sequence
  Description of Artificial Sequence Synthetic oligonucleotide
accgctgtcaacaagcagc

SEQ ID NO: 82 - DNA, 18 bp, Artificial Sequence
  Description of Artificial Sequence Synthetic oligonucleotide
tcctagatggaccgctgt

SEQ ID NO: 83 - DNA, 16 bp, Artificial Sequence
  Description of Artificial Sequence Synthetic oligonucleotide
taacacactgtcctag

SEQ ID NO: 84 - DNA, 18 bp, Artificial Sequence
  Description of Artificial Sequence Synthetic oligonucleotide
gctgatgacagtgtttct

SEQ ID NO: 85 - DNA, 18 bp, Artificial Sequence
  Description of Artificial Sequence Synthetic oligonucleotide
gcctcactctcgctcctg

SEQ ID NO: 86 - DNA, 18 bp, Artificial Sequence
  Description of Artificial Sequence Synthetic oligonucleotide
ggactgtatctggactgc

SEQ ID NO: 87 - DNA, 18 bp, Artificial Sequence
  Description of Artificial Sequence Synthetic oligonucleotide
gtggacagtcatgtatat

SEQ ID NO: 88 - DNA, 18 bp, Artificial Sequence
  Description of Artificial Sequence Synthetic oligonucleotide
tcctagatggaccgctgt

SEQ ID NO: 89 - RNA, 21 nt, Artificial Sequence
  Description of Artificial Sequence Synthetic siRNA sequence
caggugucuaggaauucgcuu

SEQ ID NO: 90 - RNA, 21 nt, Artificial Sequence
  Description of Artificial Sequence Synthetic siRNA sequence
cugugcagucacucaccaguu

SEQ ID NO: 91 - RNA, 21 nt, Artificial Sequence
  Description of Artificial Sequence Synthetic siRNA sequence
cuagguuggggucgcaguauu